(19) United States
(12) Patent Application Publication
(10) Pub. No.: US 2025/0381530 A1
(43) Pub. Date: Dec. 18, 2025

(54) IMPROVED APPARATUS FOR COMPOUND SEPARATION

(71) Applicant: LIHME PROTEIN SOLUTIONS APS, Ballerup (DK)

(72) Inventors: Allan Otto Fog LIHME, Ballerup (DK); Marie Bendix HANSEN, Ballerup (DK); Bodil Kjær LINDVED, Ballerup (DK); Peter Fog LIHME, Ballerup (DK); Dennis Kim HANSEN, Ballerup (DK); Oliver Benjamin STIGSBY, Ballerup (DK)

(21) Appl. No.: 18/880,939

(22) PCT Filed: Jun. 26, 2023
(86) PCT No.: PCT/DK2023/050164

§ 371 Date: Jan. 3, 2025

(30) Foreign Application Priority Data
Jul. 5, 2022 (DK) PA 2022 70361

(51) Int. Cl. B01D 71/26; A23C 9/142; A23C 9/146; B01J 41/09

(52) U.S. Cl. CPC B01D 71/262; A23C 9/142; A23C 9/146; B01J 41/09

(57) ABSTRACT

The present invention relates to an improved apparatus and process for separation of compounds from a biological composition by means of a dynamic layer of sorption material.

### TECHNICAL FIELD OF THE INVENTION

The present invention relates to separation of biomolecules from compositions. In particular, the present invention relates to an apparatus for collective sorption and filtration of protein.

### BACKGROUND OF THE INVENTION

Tangential flow filtration is a rapid and efficient method for separation and purification of biomolecules. Tangential flow filtration is also known as crossflow filtration. Tangential flow filtration is characterised by a majority of the feed flow traveling tangentially across the surface of the filter. The main driving force of cross-flow filtration is transmembrane pressure, which is a measure of the pressure difference between the two sides of the membrane. During the feed being passed across the filter membrane a proportion of the material being smaller than the membrane pore size will pass through the membrane as permeate, while the remaining material on the feed side of the membrane is considered as retentate.

During the separation process of biomolecules by tangential flow filtration, the biomolecules may also be bound by adsorbents in order to retain them in the retentate. State of the art is to allow the adsorbent to suspend and recirculate in the retentate and keep the tangential flow filtration membrane free from fouling and build-up of particle layers by applying a high tangential flow and frequent back flushes. However, this may leave varying amounts of target protein/target compound unbound in the composition, which may pass through to the permeate as a loss of product. A fraction of the target compounds are thus frequently wasted during the separation process. This is not optimal from a cost and resource perspective. Also, due to the suspended and recycled state of the adsorbent, considerable amounts of liquids e.g. for the washing and elution process is used in the apparatuses and methods used today for separation. In times of sparse resources, an optimisation of this part of the process would be advantageous.

Hence, an improved apparatus for separation and purification of biomolecules such as proteins would be advantageous, and in particular a more efficient and/or economic process for separation and purification of biomolecules such as proteins would be advantageous.

### SUMMARY OF THE INVENTION

Thus, an object of the present invention relates to the provision of an apparatus and a process, which optimises the amount of biomolecules, such as proteins, obtained from the separation as well as saves resources during the separation process.

In particular, it is an object of the present invention to provide an apparatus and process that solves the above mentioned problems using tangential fluid flow filtration for separation and purification.

Thus, one aspect of the invention relates to an apparatus comprising a first inlet; a tangential flow filtration unit comprising a membrane having a retentate site and a permeate site and a nominal pore size in the range of 1 to 1000 μm; one or more first fluid connections leading fluid from said first inlet to said tangential flow filtration unit; one or more second fluid connections leading retentate from said tangential flow filtration unit to said first inlet and/or said first fluid connections; a circulation pump for re-circulation of fluid through said tangential flow filtration unit via said first and second fluid connections; one or more third fluid connections for leading permeate to a first outlet; and a sorption material, wherein said sorption material comprising chemically modified porous particles having an average particle size of at least 5 micron, such as at least 10 micron, such as at least 20 micron; and said apparatus, when in use, comprises a dynamic layer of said sorption material on the retentate side of said membrane, said dynamic sorption material layer being capable of reaching a thickness of at least 1 mm, such as at least 3 mm, such as at least 5 mm, such as at least 10 mm, such as at least 15 mm, such as at least 20 mm, such as at least 25 mm, such as at least 30 mm, such as at least 35 mm, such as at least 40 mm.

Another aspect of the present invention relates to an apparatus comprising a first inlet; a tangential flow filtration unit comprising a membrane having a retentate site and a permeate site and a nominal pore size in the range of 1 to 1000 μm; one or more first fluid connections leading fluid from said first inlet to said tangential flow filtration unit; one or more second fluid connections leading retentate from said tangential flow filtration unit to said first inlet and/or first fluid connections; a circulation pump for re-circulation of fluid through said tangential flow filtration unit via said first and second fluid connections; one or more third fluid connections for leading permeate to a first outlet; wherein said apparatus further comprises a permeate pump capable of creating a partial vacuum on the permeate site of the membrane and a valve for inlet or outlet of air to the permeate site of the membrane; whereby the tangential flow filtration unit may be substantially drained or substantially filled with liquid on the permeate site of the membrane by selective controlling of the valve, the permeate pump and the circulation pump; and the tangential flow filtration unit is adapted for acquiring, when in use, a dynamic layer of a sorption material with a thickness of at least 1 mm, such as at least 3 mm, such as at least 5 mm, such as at least 10 mm, such as at least 15 mm, such as at least 20 mm, such as at least 25 mm, such as at least 30 mm, such as at least 35 mm, such as at least 40 mm on the retentate site of the membrane.

Yet another aspect of the present invention is to provide a process for separation of a target compound comprised in a biological composition, the process comprising the steps of:
i) adding to a tangential flow filtration unit comprising a membrane having a retentate site and a permeate site:
   a composition comprising a sorption material capable of adsorbing a target compound, or
   a mixture comprising a sorption material mixed with a biological composition comprising a target compound, whereby the target compound is adsorbed by the sorption material;
ii) filtrating the composition or the mixture to obtain a retentate comprising the sorption material, optionally with adsorbed target compound, and a permeate,
iii) recirculating at least part of the retentate on the tangential flow filtration unit, gradually forming a dynamic layer of said sorption material, optionally with adsorbed target compound, on the retentate site of the membrane during the recirculation of the retentate,
iv) optionally, adding a biological composition comprising target compound, filtrating the biological composition through the dynamic layer of sorption material and recirculating the retentate, whereby the target compound is adsorbed by the sorption material;
v) adding an elution liquid to release the target compound from the sorption material, and filtrating the retentate using the tangential flow filtration unit to obtain an eluted retentate comprising the sorption material and a target-containing permeate comprising the target compound;
vi) optionally, collecting the target-containing permeate and/or the permeate of steps ii)-iv) as separated product composition(s).

### BRIEF DESCRIPTION OF THE FIGURES

FIG. 1 shows, (A) a schematic representation of an apparatus according to an embodiment of the present invention, which comprises, a first inlet (1) being in fluid connection with a tangential flow filtration unit (3) through a first fluid connection (11). A second fluid connection (13) leads the retentate back to the first inlet (1), whereas a third fluid connection (17) is leading permeate to a first outlet (18). The tangential flow filtration unit (3) comprises a membrane (5) having a retentate side (7) and a permeate side (9). Furthermore, a circulation pump (15) is positioned in the first fluid connection (11); (B) a schematic representation of the circulating fluid flow over the membrane (5) of the tangential flow filtration unit (3) indicating the retentate side (7) and permeate side (9). Furthermore, a dynamic layer of sorption material is illustrated by means of several sorption particles (31) forming the dynamic layer reaching a certain thickness (32).

FIG. 2 shows a schematic representation of an apparatus according to an embodiment of the present invention, where the apparatus further comprises a permeate pump (19) in the third fluid connection (17) and a valve (21) on the permeate side (9) of the membrane (5).

FIG. 3 shows a schematic representation of an apparatus according to an embodiment of the present invention, where the apparatus further comprises a mixing tank (37).

FIG. 4 shows a schematic representation of an apparatus according to an embodiment of the present invention, where the apparatus further comprises an opening-closing valve (23), a first variable flow control valve (27) and a second variable flow control valve (29) as well as an electronic control unit (25).

FIG. 5 shows a schematic representation of an apparatus according to an embodiment of the present invention, where the apparatus further comprises a mixing tank (37), a permeate pump (19) in the third fluid connection (17) and a two-way valve (39) controlling the flow of permeate via a fourth connection (33) to the mixing tank (37).

FIG. 6 shows the fraction of adsorbent layered in the filter as a function of the permeate/retentate flow rate ratio.

FIG. 7 shows the thickness of adsorbent layer as a function of permeate/retentate flow rate ratio.

FIG. 8 shows a schematic representation of a tangential flow filtration unit having a membrane (5) in the shape of a filter tube, an inlet and an outlet for the retentate as well as a permeate outlet (45). A permeate compartment (41) is formed between the filter house (43) and the filter tube. The permeate is filtrated into the permeate compartment (41). Further an air inlet valve (21) is illustrated.

The present invention will now be described in more detail in the following.

### DETAILED DESCRIPTION OF THE INVENTION

### Definitions

Prior to discussing the present invention in further details, the following terms and conventions will first be defined:

Sorption material

In the present context, the term “sorption material” refers to a material that is able to attach to a specific compound or group of compounds, such as, but not limited to proteins. A sorption material may also be named an adsorbent.

In a preferred embodiment, the sorption material is a protein sorption material.

### Adsorbent

In the present context, the term “adsorbent” refers to a material, which is able to interact with a specific compound or group of compounds which are thereby reversibly attached to the surface of the material. For example, the adsorbent may comprise ligands able to interact with the specific compound, such as but not limited to: ion exchange ligands, hydrophobic ligands, metal chelating ligands, affinity sorption ligands, and mixed mode ligands. An adsorbent may be substantially impermeable whereby only the outer surface is available for interaction with compounds, or it may be highly porous with pores large enough to allow compounds to diffuse into and interact with the inner surface of the adsorbent.

Alternatively, the adsorbent may comprise an immobilized enzyme which is bound by reversible adsorption, physical entrapment or by covalent chemical bonding to the sorption material and for enzymatic modification of the compound present in the composition, such as but not limited to controlled hydrolysis of proteins in the composition into peptides by an immobilized protease, or modification of carbohydrates e.g. polysaccharides.

### Chemically Modified Porous Particles

In the present context, the term “chemically modified porous particles” refers to natural or synthetic porous particles being treated with one or more chemical derivatisation steps to provide porous particles with desired chemical and/or physical properties e.g. as described herein. The term also includes porous particles produced themselves by chemical reactions leading directly to porous particles having the desired chemical and/or physical properties.

### Dynamic Layer of Sorption Material

In the present context, the term “dynamic layer of sorption material” refers to a layer of sorption material on the retentate site of the membrane. This layer is dynamic as the thickness of the layer may change over time i.e. during the event of running the separation process.

The thickness of the dynamic layer may vary over the span of the retentate site of the filter. Thus, the thickness of the dynamic layer is to be understood as the average thickness reached by at least a part of the dynamic layer, such as the average thickness reached over the entire retentate site surface of the filter.

### Elution Liquid

In the present context, the term “elution liquid” refers to a liquid substance or composition, which is capable of releasing a compound attached to a sorption material.

### Circulating Flow

In the present context, the term “circulating flow” refers to the flow of the part of the fluid that enters the tangential flow filtration unit and leaves it as retentate that is circulated back to the tangential flow filtration unit, such as through the mixing tank.

### Tangential Flow Filtration Unit

In the present context, the term “tangential flow filtration unit” or cross-flow filtration unit, refer to filtration equipment wherein the filter surface is tangential to the flow direction of the composition, which is to be filtered.

### Membrane

In the present context, the term “membrane” refers to a filter or a membrane useful for separating components of a composition.

Membrane and filter are used interchangeably herein.

### Flux

In the present context, the term “flux” or “flux rate” means the volume of liquid passing one square meter of filter area per hour. The unit applied in the art is LMH (i.e. L/m²/h).

### Nominal Pore Size

In the present context, the term “nominal pore size” relates to the pore size of a filter at which a minimum percentage of components larger than that pore size should be retained by the filter. For example, a nominal pore size of 60% for a filter having an average pore size of X, is considered to retain at least 60% of particles which are larger than X.

### Retentate

In the present context, the term “retentate” relates to a composition which has passed over a filter without passing through the pores of the filter to the permeate side. Thus, a retentate is the part of a filtered composition which was retained by the filter i.e. retained on the retentate side of the filter.

### Fluid Flow of Retentate

In the present context, the term “fluid flow of retentate” relates to the flow of the retentate when entering the tangential flow filtration unit. Thus, if the retentate is not re-circulated or not fully re-circulated, this fluid flow would relate to the fluid flow of the mixture or biological composition when entering the tangential flow filtration unit.

### Permeate

In the present context, the term “permeate” relates to a composition which has passed through the pores of a filter. Thus, a permeate is the part of a filtered composition which went through the filter i.e. passed through the filter to the permeate side of the filter.

### Fluid Flow of Permeate

In the present context, the term “fluid flow of permeate” relates to the flow of permeate measured either in the third fluid connection or at the first outlet.

### Continuous Tangential Fluid Flow

In the present context, the term “continuous tangential fluid flow” relates to having a tangential flow throughout all the process steps.

### Backward Flush

In the present context, the term “backward flush”, or “back flush”, relates to an event wherein the flow of composition through the pores of the filter is reversed i.e. from the permeate site of the membrane to the retentate site.

Pulsed Pressure Increase and/or Backward Flush of the Permeate

In the present context, the term “pulsed pressure increase and/or backward flush of the permeate” relates to an event wherein the pressure on the permeate side is increased. For example, the pressure increase may be large enough that the difference between the pressure on the retentate side and the pressure on the permeate side of the filter is essentially zero, whereby the flow of composition through the filter may be reversed to give a weak backward flush though the filter. In another example, the pressure increase on the permeate side is much larger than the pressure on the retentate side, whereby a strong backward flush is applied.

### Biological Composition

In the present context, the term “biological composition” refers to a complex composition obtained from a biological source, such as plants, microorganisms and animals, and being a liquid, suspension or blend. For example, the biological composition may be milk, whey, fermentation broth, plant extracts or blood plasma.

### Target Compound

In the present context, the term “target compound” refers to the compound in the biological composition, which is to be separated from the biological composition.

The target compound may be any compound of interest such as coloured molecules, nucleotides, polyphenols, metal ions, toxic compounds and biomolecules such as proteins and peptides.

In a preferred embodiment, the target compound is a target protein.

### Mixture

In the present context, the term “mixture” refers to a biological composition comprising a target compound which is mixed with sorption material.

### Refining

In the present context, the term “refining” refers to a process wherein a compound is concentrated, separated, cleaned, purified, and/or isolated from a biological composition.

### Separation

In the present context, the term “separated” refers to something which has been set apart from something else. More specifically, it may be a compound which has been set apart from other species of a composition, such that a new composition comprising the compound is obtained and another new composition not comprising the compound is obtained. In some applications the new composition not comprising the species bound to the adsorbent may (also) be a/the target product.

### Transmembrane Pressure (TMP)

In the present context, the term “transmembrane pressure” is defined as:

$$TMP = ( feed pressure + retentate pressure ) / 2 - permeate pressure .$$

### Apparatus

The present invention provides an apparatus for refining a compound comprised in a biological composition.

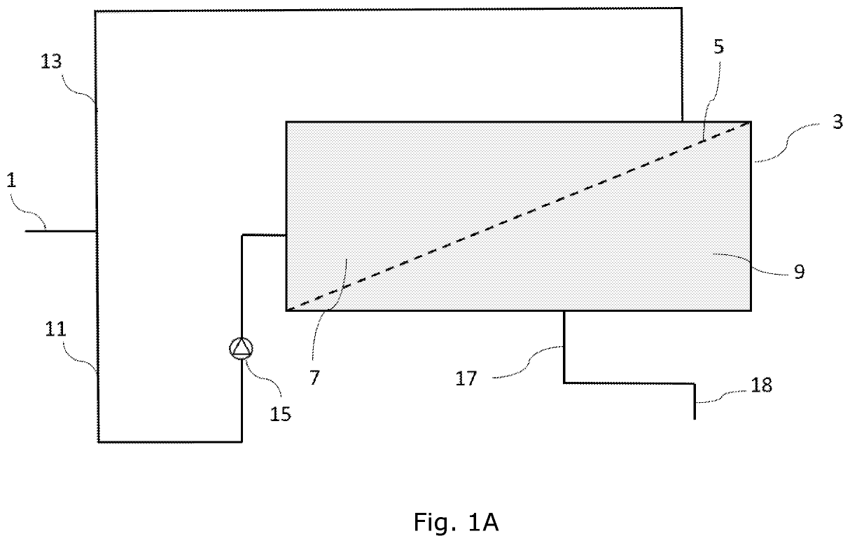

By reference to FIG. 1A but without being limited hereby, a first aspect of the present invention relates to an apparatus comprising a first inlet (1); a tangential flow filtration unit (3) comprising a membrane (5) having a retentate site (7) and a permeate site (9) and a nominal pore size in the range of 1 to 1000 μm; one or more first fluid connections (11) leading fluid from said first inlet (1) to said tangential flow filtration unit (3); one or more second fluid connections (13) leading retentate from said tangential flow filtration unit (3) to said first inlet (1) and/or said first fluid connections (11); a circulation pump (15) for re-circulation of fluid through said tangential flow filtration unit (3) via said first and second fluid connections (11,13); one or more third fluid connections (17) for leading permeate to a first outlet (18); and a sorption material, wherein said sorption material comprising chemically modified porous particles having an average particle size of at least 5 micron, such as at least 10 micron, such as at least 20 micron; and said apparatus, when in use, comprises a dynamic layer of said sorption material on the retentate side (7) of said membrane (5), said dynamic sorption material layer being capable of reaching a thickness of at least 1 mm, such as at least 3 mm, such as at least 5 mm, such as at least 10 mm, such as at least 15 mm, such as at least 20 mm, such as at least 25 mm, such as at least 30 mm, such as at least 35 mm, such as at least 40 mm.

FIG. 1 shows the circulation pump (15) being arranged in the first fluid connection (11). However, the circulation pump (15) may as well be arranged in the second fluid connection (13) as long as the circulation pump (15) is adapted for re-circulating the retentate in the apparatus. Thus, in one embodiment, the circulation pump (15) is arranged in the first fluid connection (11). In a further embodiment, the circulation pump (15) is arranged in the second fluid connection (13).

The apparatus forms a dynamic layer of the sorption material on the retentate side of the membrane once the process is initiated. The layer is gradually formed during the process. Forming a layer of sorption material results in a more efficient separation process as well as a process demanding less resources for washing and elution. FIG. 1B illustrates the formation of a dynamic layer of sorption material on the retentate side (7) of the membrane (5). The dynamic layer is formed by sorption particles (31) reaching a given thickness (32).

Alternatively, said dynamic layer could be present in the apparatus prior to use having been formed during a previous run.

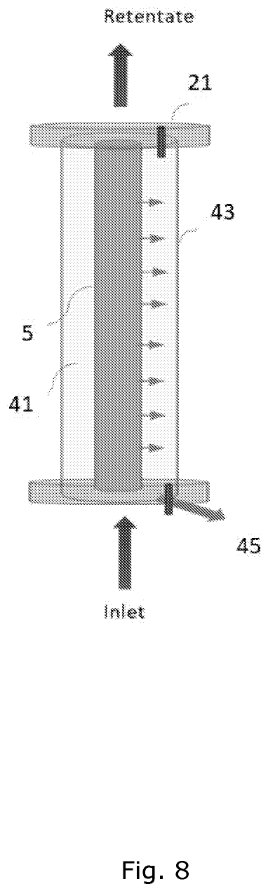

The tangential flow filtration unit (3) may be equipped with any type of membrane (5). The third fluid connection (17) leading permeate to outlet is connected to the first outlet (18) that may direct the permeate for collection in a waste tank or in a permeate collection tank. It is to be understood that in one embodiment, the outlet is to be understood merely as an opening from which permeate may leave the apparatus, e.g. as shown in FIG. 8.

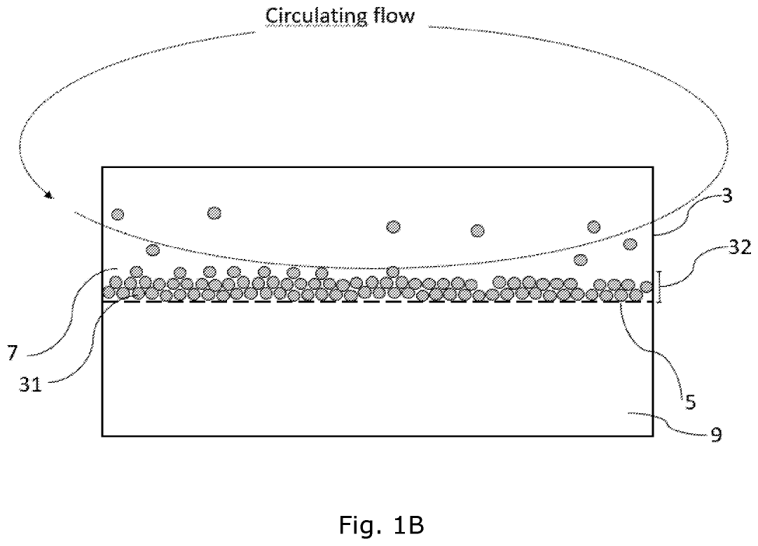

FIG. 2 shows a more detailed schematic representation of an apparatus according to an embodiment of the present invention. In this embodiment, a permeate pump (19) is inserted in the third fluid connection (17) for controlling the fluid flow of the permeate. Hence, in one embodiment, the apparatus further comprises a permeate pump (19). In a further embodiment, the permeate pump (21) is arranged in connection with the one or more third fluid connections (17).

The embodiment, as shown in FIG. 2, further shows a valve (21) inserted on the permeate site (9) of the tangential flow filtration unit (3). This valve (21) is able to let air into or out of the permeate site (9) of the membrane (5) in order to obtain an evenly distributed partial vacuum on the permeate site (9) of the membrane (5), when so wished for. Hence, in one embodiment, the apparatus further comprises a valve, where the valve (21) is capable of inlet or outlet of air to the permeate site (9) of the membrane (5). In a further embodiment, the valve (21) is arranged in connection with the tangential flow filtration unit (3).

FIG. 2 also shows an embodiment of a further aspect of the present invention. The further aspect of the present invention relates to an apparatus comprising a first inlet (1); a tangential flow filtration unit (3) comprising a membrane (5) having a retentate site (7) and a permeate site (9) and a nominal pore size in the range of 1 to 1000 μm; one or more first fluid connections (11) leading fluid from said first inlet (1) to said tangential flow filtration unit (3); one or more second fluid connections (13) leading retentate from said tangential flow filtration unit (3) to said first inlet (1) and/or first fluid connections (11); a circulation pump (15) for re-circulation of fluid through said tangential flow filtration unit (3) via said first and second fluid connections (11,13); one or more third fluid connections (17) for leading permeate to a first outlet (18); wherein said apparatus further comprises a permeate pump (19) capable of creating a partial vacuum on the permeate site (9) of the membrane (5) and a valve (21) for inlet or outlet of air to the permeate site (9) of the membrane (5); whereby the tangential flow filtration unit (3) may be substantially drained or substantially filled with liquid on the permeate site (9) of the membrane (5) by selective controlling of the valve (21), the permeate pump (19) and the circulation pump (15); and the tangential flow filtration unit (3) is adapted for acquiring, when in use, a dynamic layer of a sorption material with a thickness of at least 1 mm, such as at least 3 mm, such as at least 5 mm, such as at least 10 mm, such as at least 15 mm, such as at least 20 mm, such as at least 25 mm, such as at least 30 mm, such as at least 35 mm, such as at least 40 mm on the retentate site (7) of the membrane (5).

In one embodiment, the permeate pump (21) is arranged in connection with the one or more third fluid connections (17). In a further embodiment, the valve (21) is arranged in connection with the tangential flow filtration unit (3).

It is important for the advantageous functioning of the system that the tangential flow filtration unit holds the ability to form a dynamic layer of sorption material during the process. Thus, the tangential flow filtration unit needs to be adapted therefore. To be able to form a dynamic layer of sorption material, sorption material needs to be present. The sorption material may either be provided by the initiation of or during the separation process to be run on the apparatus or it may be present in the apparatus prior to running of the process. Thus, in one embodiment, the apparatus further comprises a sorption material. In a further embodiment, the sorption material is porous.

The details relating to the valve (21) for letting air into the permeate site of the filter is further detailed in the embodiment shown in FIG. 8. In this figure a single filter house (43) is illustrated showing how fluid enters the filter house (43) at the ‘inlet’ and exits as ‘retentate’. The membrane (5) filtrates the fluid and permeate is entered (arrows) into the permeate compartment (41) and exits via the permeate outlet (45).

The permeate site (9) of the membrane (5) may hereby be substantially drained or substantially filled with liquid. By being substantially drained is understood that at least 50%, such as at least 60%, such as at least 70%, such as at least 75%, such as at least 80%, such as at least 85%, such as at least 90%, such as at least 95% of the permeate compartment volume (41) on the permeate side of the tangential flow filtration unit is filled with air. By being substantially filled with liquid is understood that at least 50%, such as at least 60%, such as at least 70%, such as at least 75%, such as at least 80%, such as at least 85%, such as at least 90%, such as at least 95% of the permeate compartment volume (41) on the permeate side of the tangential flow filtration unit is filled with liquid.

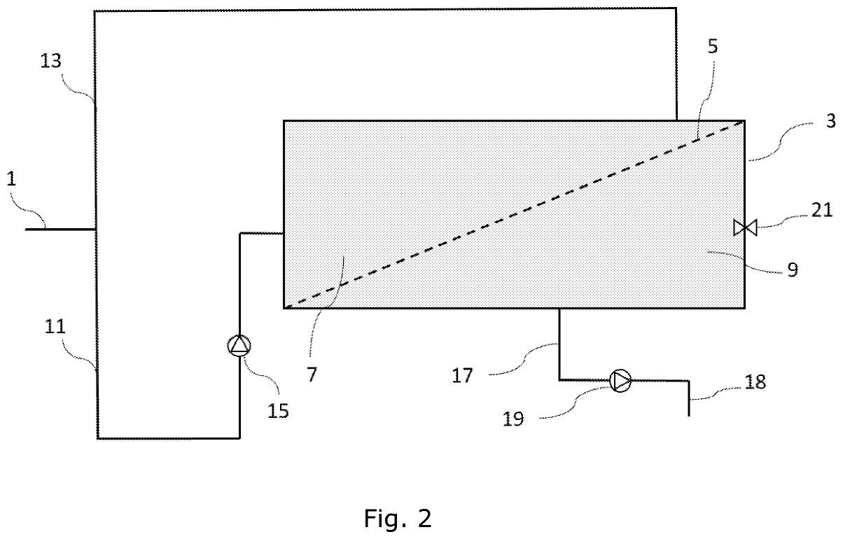

The apparatus according to the invention may comprise different add-ons. One of such could be a mixing tank (37) in which a biological composition and the sorption material could be mixed prior to being filtrated. However, the mixing tank may also be used just for the purpose of storing a mixture for addition hereof to the tangential flow filtration unit. An apparatus comprising a mixing tank (37) is shown in FIG. 3, which resembles FIG. 1 except for the mixing tank (37). As shown in the embodiment illustrated in FIG. 3, the mixing tank (37) is arranged in fluid connection with the first fluid connection (11) and with the first inlet (1) being an inlet to the mixing tank (37).

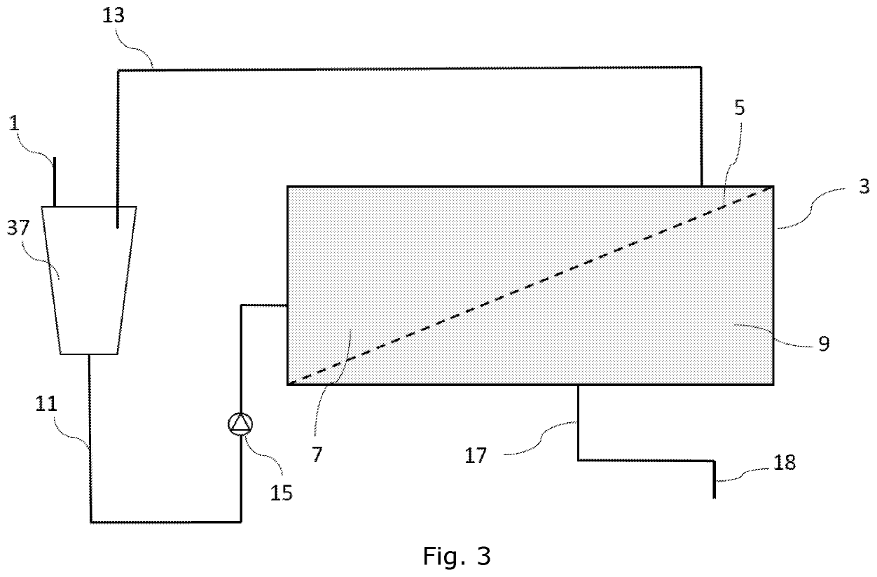

A further schematic representation of an apparatus according to an embodiment of the present invention is shown in FIG. 4 and relates to an apparatus as illustrated in FIG. 1, but wherein the apparatus is equipped with a first variable flow control valve (27) in the second fluid connection (13) and a second variable flow control valve (29) in the third fluid connection (17); both valves being controlled by one or more electronic control units (25). The variable flow control valves may be used in conjunction with the opening-closing valve to create a particularly controllable pulsed pressure increase on the permeate side of the filter, such as to obtain a modest backward flush of the permeate.

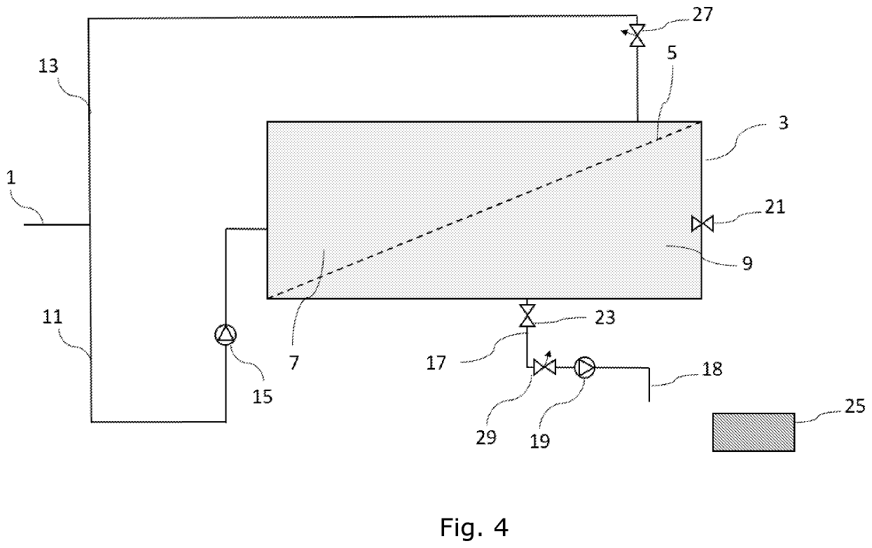

A further schematic representation of an apparatus according to an embodiment of the present invention is shown in FIG. 5 and relates to an apparatus as illustrated in FIG. 3, but wherein the apparatus further comprises a permeate pump (19). In addition, this embodiment comprise a fourth fluid connection (33), which transports permeate via the third fluid connection (17) to the mixing tank (37), where it is recirculated over the tangential flow filtration unit (3). The flow from the third fluid connection (17) into the fourth fluid connection (33) is controlled by a two-way valve (39). The embodiment as illustrated in FIG. 5 was used for the experiments.

### Sorption Material

The sorption material is important for the formation of the dynamic layer. The sorption material may be chemically modified porous particles. Thus, in one embodiment, the sorption material is chemically modified porous particles.

The shape of the sorption material can be of influence on the packaging of the dynamic layer. The sorption material in one embodiment comprises irregular particles, which will allow fluid to pass through the layer to the membrane and for the target compound to reach the particles to be adsorbed hereby. In a further, preferred, embodiment, the sorption material is spherical.

The sorption material is preferably porous. The pores influence the absorption of the target compound by the sorption material as it allows migration and binding of compounds to the material surfaces presented inside the volume of the particles. Porosity may be defined as the total pore volume of all pores with a diameter smaller than 150 nm is between 0.5 ml/g and 1.5 ml/g, such as in the range 0.7-1.1 ml/g. Porosity, with respect to this invention, is also determined by mercury intrusion.

In one embodiment, at least 90%, such as 95%, such as 98% and preferably at least 99% of said sorption material have a lower pore diameter limit of 10 nm and an upper pore diameter limit of 200 nm. In a further embodiment, the total pore volume of all pores with a diameter smaller than 150 nm is between 0.5 ml/g and 1.5 ml/g, such as in the range 0.7-1.1 ml/g.

The size of the sorption material is also influential on the dynamics of the layer. In an embodiment, the sorption material has a size distribution such that 90% by volume of the particles have a diameter in the range of 10-1000 μm, such as in the range of 10-700 μm, such as in the range of 10-500 μm, such as in the range of 10-400 μm, such as in the range of 10-300 μm such as in the range of 10-250 um, such as in the range of 10-200 μm, such as in the range of 10-150 μm, such as in the range of 10-100 μm, such as in the range of 10-80 μm, such as in the range of 10-70 μm, such as in the range of 10-60 μm, such as in the range of 10-50 μm, such as in the range of 10-40 μm.

In a further embodiment, the sorption material has a size distribution such that 90% by volume of the particles have a diameter in the range of 30-1000 μm, such as in the range of 30-700 μm, such as in the range of 30-500 μm, such as in the range of 30-400 μm, such as in the range of 30-300 μm, such as in the range of 30-250 μm, such as in the range of 30-200 μm, such as in the range of 30-150 μm, such as in the range of 30-100 μm, such as in the range of 30-90 μm, such as in the range of 30-70 μm; like in the range of 40-250 μm, such as in the range of 50-250 μm, such as in the range of 50-200 μm, such as in the range of 50-150 μm.

The density of the sorption material is also of importance for the formation of a dynamic layer being able to stay in the layered state at the preferred flow conditions where the fluid flows of the permeate and retentate are adjusted to one another as described herein. Thus, the sorption material preferably has a density in the wet state in the range of 1.00 to 1.50 g/ml, such as in the range of 1.01 to 1.40 g/ml, such as in the range of 1.02 to 1.30 g/ml, such as in the range of 1.02 to 1.20 g/ml, such as in the range of 1.02 to 1.10 g/ml, such as in the range of 1.02 to 1.08 g/ml, such as in the range of 1.02 to 1.06 g/ml.

In another embodiment the sorption material has a density in the wet state in the range of 1.01 to 1.10 g/ml, such as in the range of 1.01 to 1.08 g/ml, such as in the range of 1.01 to 1.06 g/ml.

In a preferred combination, the sorption material is spherical, with a particle diameter in the range of 30-300 μm, such as 50-200 μm, and a density in the range of 1.01 to 1.10 g/ml.

In a further embodiment, the sorption material is spherical with a diameter in the range of 10-60 μm, such as in the range of 10-50 μm, such as in the range of 10-40 μm and a density in the range of 1.01 to 1.10 g/ml.

In order for the compounds, such as proteins, of the biological composition to be attached to the sorption material, said material should have surface characteristics that enables efficient interaction with compound molecules. The base material of the sorption material may have suitable binding sites (ligands) already present when the sorption material particles are formed, or the ligands may be introduced and covalently coupled to the material of particles after the initial formation.

The type of sorption material to be utilized depends on the nature of the compound to be separated from the biological composition. The sorption material should preferably get attached to the target compound to form a conjugate-like structure (such as by adsorption). The conjugate carries the compound around in the circulating flow, such that the sorption material conjugate passes over the retentate side of the filter several times and/or be maintained in the dynamic layer to allow impurities in the circulation flow to pass through the filter to the permeate side and into a waste tank or permeate collection tank. The sorption material conjugate may be further cleaned and isolated by use of an installed washing system applying one or more washing liquids to the circulating flow. Whenever, a desired concentration and purity have been reached an elution liquid is added to the circulating flow, whereby the target compound is released from the sorption material. The liquid composition comprising the target compound then passes through the filter to the permeate side and may then be collected from an outlet or collected in the permeate collection tank.

### Material

In one embodiment of the present invention, the sorption material is an inorganic sorption material, an organic polymer sorption material or a composite of inorganic and organic polymer materials.

During use of the apparatus, the sorption material will often be exposed to a wide range of pH values, harsh chemicals and even high temperatures. It is therefore important that the sorption material is stable under such conditions and that it does not erode into smaller particles, gets solubilized, or obtains an altered and undesired porous structure. For example, the sorption material should preferably be resistant to shear forces. Such a resistant and stable sorption material may be obtained through chemical cross-linking of the sorption material backbone by methods generally used for stabilization of chromatographic adsorbents. Thus, an embodiment of the present invention relates to the sorption material being cross-linked.

Examples of suitable cross-linking agents are epichlorohydrin, boric acid, epibromohydrin, allyl bromide, allyl glycidylether; bis-epoxides such as butanediol diglycidylether; halogen-substituted aliphatic compounds such as di-chloro-propanol, divinyl sulfone, diacrylates, diacrylamides such as piperazine-diacrylamide, bis-acrylamides such as N,N-methylene-bisacrylamide, and mixtures and derivatives thereof. A particular embodiment of the present invention relates to the apparatus as described herein, wherein the sorption material has been cross-linked with a reagent selected from the group consisting of epichlorohydrin, butanediol diglycidylether, diacrylates, bis-acrylamide, allyl bromide, allyl glycidylether, boric acid, and mixtures and derivatives thereof.

The sorption material may comprise, as the backbone, any natural or synthetic and organic or inorganic material known per se to be applicable in adsorption based separation of preferably proteins, e.g. natural or synthetic polysaccharides such as agar-agar and agaroses; celluloses, cellulose ethers such as hydroxypropyl cellulose, carboxymethyl celluose; starches; gums such as guar gum, and gum arabic, gum ghatti, gum tragacanth, locust bean gum, xanthan gum; pectins; mucins; dextrans; chitins; chitosans; alginates; carrageenans; heparins; gelatins; synthetic polymers such as polyamides such as polyacrylamides and polymethacrylamides; polyimides; polyesters; polyethers; polymeric vinyl compounds such as polyvinylalcohols and polystyrenes; polyalkenes; inorganic materials such as silicious materials such as silicon dioxide including amorphous silica and quartz; silicas; metal silicates, controlled pore glasses and ceramics; metal oxides and sulfides, or combinations of these natural or synthetic and organic or inorganic materials.

An embodiment of the present invention relates to the sorption material being a polymer sorption material comprising one or more polymers selected from the group consisting of natural polysaccharides, agar-agar, agarose, dextran, cellulose, cellulose ethers, hydroxypropyl cellulose, carboxymethyl celluose, starches, gum, guar gum, gum arabic, gum ghatti, gum tragacanth, locust bean gum, xanthan gum, pectin, mucin, chitin, chitosan, alginate, carrageenan, heparin, gelatin, synthetic polymer, polyamide, polyacrylamide, polymethacrylamide, polyimide, polyacrylate, polymeric vinyl, polystyrene, polyalkene, polyvinyl alcohol, polyester, polyether, silicious material, silicon dioxide, amorphous silica, quartz, silica, metal silicate, controlled pore glass, ceramic, metal oxide, sulphide, and composites thereof.

Especially interesting materials as matrix backbones for the sorption material are e.g. agar or agarose beads such as Sepharose and Superose beads from Cytiva, USA, Agarose Beads from ABT, Spain and Biogel A from Biorad, USA; dextran based beads such as Sephadex, Cytiva, USA; cellulose based beads and membranes such as Perloza cellulose Czech Republic, composite beads such as Sephacryl and Superdex, Cytiva, USA; beads of synthetic organic polymers such as Fractogel from Toso-Haas, USA.

A particular embodiment of the present invention relates to a sorption material being a polymer sorption material comprising one or more polymers selected from the group consisting of natural polysaccharide, agar-agar, agarose, dextran, cellulose, gum, pectin, synthetic polymer, polyacrylate, polyvinyl alcohol, polyester, polyether, and mixtures and/or chemical derivatives thereof.

In a further embodiment, the sorption material is an inorganic material selected from the group consisting of porous glass, porous silica, porous ceramics, clays and composites of these with organic polymers.

In yet another embodiment, the sorption material is an ion exchange sorption material comprising acidic moieties or basic moieties. In a further embodiment, the acidic moieties are selected from the group consisting of carboxylic acid, phosphoric acid and sulfonic acid. In another embodiment, the basic moieties are selected from the group consisting of primary, secondary, tertiary or quaternary amines.

In a further embodiment, the sorption material is an affinity sorption material. In a still further embodiment, the sorption material is an affinity sorption material selected from the group consisting of metal chelate adsorption material, hydrophobic interaction adsorption material, protein A adsorption material, and immobilized antibody adsorption material.

The sorption material may have been obtained by reacting the sorption material used as the backbone for the sorption material with any compound that after reaction provides a sorption material having ligands that interact, such as may bind to the target compound to be separated from the biological composition.

Such interactions are, for example, the presence of negatively or positively charged groups covalently linked to, or being a natural integral part of the sorption material. Examples of groups that may form negatively charged groups, depending on pH in the medium, are carboxylic acids, sulfonic acids, and phosphonic acids. Examples of groups that may form positively charged groups, depending on pH in the medium, are primary amines, secondary amines, tertiary amines such as the diethylaminoethyl-group (DEAE) and quaternary amines, such as the diethyl-(2-hydroxypropyl) aminoethyl-group (QAE). These charged sorption materials may be broadly used for separation and isolation of compounds, such as proteins and other biomolecules, by the ion exchange chromatography method. Thus, in one embodiment, the sorption material comprises ligands.

An embodiment of the present invention therefore relates to the sorption material comprising one or more type of ligand selected from the group consisting of ion exchange ligand, affinity sorption ligand, and mixed mode ligand. More specifically, an embodiment of the present invention relates to the apparatus as described herein, wherein the sorption material is an affinity sorption material. In this regard, an embodiment of the present invention relates to the sorption material being an affinity sorption material selected from the group consisting of metal chelate adsorption material, hydrophobic interaction adsorption material, thiophilic interaction adsorption material, mixed mode interaction material, protein A adsorption material, and immobilized antibody adsorption material.

Hydrophobic interactions between the sorption material and compounds to be isolated may, for example, be achieved if the material surface has hydrophobic groups such as phenyl-groups, hydrocarbons such as octyl-, hexyl or butyl-groups. Thiophilic interactions may, for example, be achieved if the material surface is activated with divinyl sulfone and coupled with mercaptoethanol or 4-hydroxy-pyridine, 3-hydroxy-pyridine, 2-hydroxy-pyridine. Mixed mode interactions may, for example, be achieved if the material surface has groups that can be either non-charged and hydrophobic or positively or negatively charged, dependent on pH in the medium, such as benzylamines; alkylamines, such as hexyl- and octyl-amines; benzoic acids, such as mercapto-benzoic acids and amino-benzoic acids; phenyl-alkyl carboxylic acids, aminophenyl-alkyl carboxylic acids.

The ligands may be attached to the sorption material by any type of bond, either by a direct chemical reaction between the ligand and the sorption material used as the backbone for the sorption material or by activation of the ligand with a suitable reagent making it possible to link the backbone material and the ligand. Examples of such suitable activating reagents are epichlorohydrin, epibromohydrin, allyl bromide, allyl glycidylether; bis-epoxides such as butanediol diglycidylether; halogen-substituted aliphatic compounds such as di-chloro-propanol, divinyl sulfone; carbonyldiimidazole; aldehydes such as glutaric dialdehyde; quinones; cyanogen bromide; periodates such as sodium-meta-periodate; carbodiimides; chloro-triazines such as cyanuric chloride; sulfonyl chlorides such as tosyl chlorides and tresyl chlorides; N-hydroxy succinimides; 2-fluoro-1-methylpyridinium toluene-4-sulfonates; oxazolones; maleimides; pyridyl disulfides; and hydrazides. Among these, the activating reagents leaving a spacer group different from a single bond, e.g. epichlorohydrin, epibromohydrin, allyl-glycidylether; bis-epoxides; halogen-substituted aliphatic compounds; divinyl sulfone; aldehydes; quinones; cyanogen bromide; chloro-triazines; oxazolones; maleimides; pyridyl disulfides; and hydrazides. Especially interesting activating reagents are believed to be epoxy-compounds such as epichlorohydrin, allyl-glycidylether and butanediol diglycidylether. A particular embodiment of the present invention relates to the apparatus as described herein, wherein the sorption material has ligands obtained by reaction with a reagent selected from the group consisting of epichlorohydrin, allyl-glycidylether, benzylamine, glycidyl-trimethyl-ammonium chloride, and butanediol diglycidylether.

The ligand concentration on the sorption material may have a strong influence on the compound binding capacity of the material. If the ligand concentration is too low then only small amounts of compound may be bound per liter of the sorption material. However, if the ligand concentration is too high it may also lead to more or less irreversible binding of the compound which will lead to unwanted product losses and poor economic performance of the process. In the context of the present invention the compound binding capacity is defined as the amount of compound (expressed as gram dry compound) that can be bound to one liter of a sorption material which is fully hydrated but drained for interstitial water by suction drying on a sintered, porous glass funnel. In an embodiment of the present invention relating to the apparatus as described herein, the sorption material has a compound binding capacity in the range of 1-250 g/L sorption material, such as 5-200 g/L, such as 10-200 g/L, such as 20-200 g/L, such as 25-200 g/L, such as 30-200 g/L, and such as 40-200 g/L.

The optimal ligand concentration may depend on the specific application, the compound concentration in the biological composition and other process specific parameters. In the context of the present invention the ligand concentration is defined as the amount of ligand expressed in millimoles (i.e. 10⁻³ mole) that is attached to one liter of a sorption material which is fully hydrated but drained for interstitial water by suction drying on a sintered, porous glass funnel.

An embodiment of the present invention relates to the ligand concentration on the sorption material being in the range of 1-3000 millimoles/L of sorption material, such as 10-2500 millimoles/L, such as 20-2500 millimoles/L, such as 30-2500 millimoles/L, such as 40-2500 millimoles/L, such as 50-2500 millimoles/L, such as 60-2500 millimoles/L, such as 80-2500 millimoles/L, such as 100-2500 millimoles/L, such as 125-2500 millimoles/L, such as 150-2500 millimoles/L, and such as 200-250 millimoles/L.

For certain applications, the compound binds very strongly to the sorption material and then the ligand concentration should be in the range of 1-300 millimoles/L. Thus, a particular embodiment of the present invention relates to the the ligand concentration on the sorption material being in the range of 1-300 millimoles/L, such as 5-250 millimoles/L, such as 10-225 millimoles/L, such as 20-200 millimoles/L, such as 30-180 millimoles/L, such as 30-150 millimoles/L, such as 30-120 millimoles/L, such as 30-100 millimoles/L, such as 30-80 millimoles/L, such as 30-60 millimoles/L. However, particularly the ligand concentration on the sorption material may be in the range of and such as 40-150 millimoles/L, 40-120 millimoles/L, 40-100 millimoles/L, 40-80 millimoles/L.

In a still further embodiment, the sorption material carries an immobilized enzyme, which is bound by reversible adsorption, physical entrapment or by covalent chemical bonding to the sorption material.

### Concentration of Sorption Material

The retentate side of the membrane unit and the fluid connections enabling recirculation of the sorption material constitutes some of the elements of the apparatus wherein the sorption material is distributed during use of the apparatus. The total volume of the fluid wherein the sorption material is distributed is herein defined as the “system volume”. The system volume is not a fixed volume since the mixing tank may be filled to different degrees and the volume may also be adjusted during operation. The sorption material will be suspended in the fluid at a concentration which is measured as the relative volume of packed sorption material achieved when a sample of the suspended material is centrifuged at 3000 G for 5 min expressed in percent of the total suspension sample volume. Thus, if a 100 ml sample of the suspended sorption material is withdrawn from a well-mixed suspension in the mixing tank and centrifuged and the packed bed of sorption material is determined to have a volume of 33 ml, then the concentration of sorption material is 33/100×100%=33%.

The total water consumption per kg compound produced is defined as the volume of aqueous fluids added to the apparatus during the washing and elution steps of the process according to the invention per kg compound (on a dry matter basis) eluted during one cycle of the process. Accordingly, the water consumption per kg compound produced will depend on the concentration of the sorption material suspended in the system volume during washing and elution. If all of the compound adsorption material is suspended in the system volume the water consumption during washing and elution will be relatively high due to the need for substitution (washing out) of the entire system volume by simple dilution. When substantially all of the sorption material is layered in the tangential flow filtration unit during washing and elution the water consumption will be relatively lower due to the plug flow (non-mixed) conditions obtained by fixing the adsorbent as a layer in the filtration unit.

During binding of the compound to the sorption material it may be advantageous to allow at least a fraction of the sorption material to be suspended and recycled in the system volume. Hereby, it may be achieved to adsorb some or all of the target compound prior to the passage through the filter membrane and any layered sorption material.

Thus, an embodiment of the present invention relates to the sorption material being suspended in an aqueous liquid. A further embodiment of the present invention relates to the sorption material is suspended in an aqueous liquid at a concentration of 5-70% by volume. In a still further embodiment, the sorption material is suspended in the aqueous liquid at a concentration of at least 0.1% by volume, such as at least 1% by volume, such as at least 5% by volume, such as at least 7% by volume such as at least 10% by volume, such as at least 15% by volume, such as at least 20% by volume, such as at least 25% by volume, such as at least 30% by volume, such as at least 35% by volume, such as at least 40% by volume, such as in the range of 5-55% by volume, such as in the range of 10-50% by volume, such as in the range of 15-45% by volume, such as in the range of 20-40% by volume.

For some applications it may further be advantageous to use different concentrations of the sorption material during the different steps of a compound adsorption cycle. Thus, an embodiment of the present invention relates to the concentration of the sorption material during loading of the mixture or the sorption material itself being in the range of 1-30%, such as in the range of 3-30%, such as in the range of 5-30%, such as in the range of 5-25%, such as in the range of 7-25%, and such as in the range of 10-25%. In another embodiment of the present invention, the concentration of the sorption material during wash and/or elution is in the range of 20-70%, such as in the range of 25-70%, such as in the range of 25-65%, such as in the range of 30-65%, and such as in the range of 40-65%.

### Compound Binding Capacity

The compound binding capacity of the sorption material is likewise of importance for the total liquid consumption per kg compound produced of any compound separation process performed with the apparatus. If the compound binding capacity is low, the volume of aqueous fluids added to the apparatus during the washing and elution steps of the process according to the invention per kg compound (on a dry matter basis) during one cycle of the process will be high, and thereby the waste handling and cost of the process may become too high.

Thus, an embodiment of the present invention relates to the sorption material having a binding capacity for the compound to be refined of at least 5 g/L adsorbent, such as at least 10 g/L adsorbent, such as at least 15 g/L, such as at least 20 g/L, such as at least 25 g/L, such as at least 30 g/L. such as at least 35 g/L, such as at least 40 g/L adsorbent on a dry matter basis.

The surface area of the membrane relative to the adsorbent volume is influencing the productivity of the apparatus in terms of the amount of compound that may be refined per litre of adsorbent per hour. If the membrane area/adsorbent volume ratio is relatively low the productivity will be low, while, if the ratio is relatively high the productivity may be at a maximum.

Thus, in an embodiment of the present invention relating to the apparatus as described herein, the membrane area relative to the volume of adsorbent is at least 0.01 m²/L, such as at least 0.02 m²/L, such as at least 0.05 m²/L, such as at least 0.08 m²/L, such as at least 0.10 m²/L, such as at least 0.15 m²/L, such as at least 0.20 m²/L.

However, for some applications the membrane area relative to the adsorbent volume may also become too large (e.g. in terms of equipment and maintenance cost for the membrane unit and the total system volume), and it is therefore an embodiment of the invention that the membrane area relative to the adsorbent volume is in the range of 0.01-0.5 m²/L, such as 0.01-0.4 m²/L, such as 0.01-0.3 m²/L, such as 0.01-0.2 m²/L, or such as in the range of 0.02-0.4 m²/L, such as 0.02-0.3 m²/L, such as 0.02-0.2 m²/L, or such as in the range of 0.03-0.4 m²/L, such as 0.03-0.3 m²/L, such as 0.03-0.2 m²/L, or such as in the range of 0.04-0.4m²/L, such as 0.04-0.3 m²/L, such as 0.04-0.2 m²/L, or such as in the range of 0.05-0.4 m²/L, such as 0.05-0.3 m²/L, such as 0.05-0.2 m²/L, such as 0.05-0.15 m²/L, such as 0.05-0.10 m²/L.

### Dynamic Layer of Sorption Material

During use of the apparatus and recirculation of the retentate, the sorption material forms a dynamic layer on the retentate site of the membrane. The layer is considered dynamic as the thickness hereof may vary during the separation process. The thickness of the dynamic layer may further be adjusted by adjusting the retentate fluid flow and the permeate fluid flow. Alternatively or additionally, the dynamic layer may be partly or fully removed by a backward flush during the process and subsequently reformed. This may e.g. be advantageous if the filter and/or the layered adsorbent shows declined flow capabilities due to fouling or entrained suspended solids from the biological composition.

In one embodiment, the dynamic layer reaches a thickness of at least 1 mm, such as at least 3 mm, such as at least 5 mm, such as at least 10 mm, such as at least mm, such as at least 20 mm, such as at least 25 mm, such as at least 30 mm, 15 such as at least 35 mm, such as at least 40 mm.

In one embodiment of the invention, the dynamic layer during the washing and/or elution steps is made up of at least 50%, such as at least 60%, such as at least 70%, such as at least 80%, such as at least 85%, such as at least 90%, such as at least 95%, such as at least 98%, such as at least 99% of the sorption material present in the apparatus.

In one embodiment of the invention, substantially all of the sorption material present in the apparatus is part of the dynamic layer during the washing and/or elution steps.

### Biological Composition/Mixture

The biological composition comprises target compounds to be purified from the composition. This is performed by adsorption of the target compound by the sorption material. The biological material may be any composition as known to persons skilled in the art. In one embodiment, the sorption material is able to adsorb one or more compounds, such as proteins, of a biological composition selected from the group consisting of bovine milk and whey, bovine blood plasma, porcine blood plasma, human blood plasma, egg white, egg yolk, fermentation broth, fermentation broth of genetically modified microorganisms, fermentation broth of mammalian cell cultures, plant tissue extracts, potato tuber extract, and legume extract.

Particularly, in one embodiment, the sorption material is able to adsorb one or more compounds of a biological composition, the biological composition being:
bovine milk or whey comprising at least one of said proteins selected from the group consisting of lactoferrin, immunoglobulin G, alpha-lactalbumin, plasminogen, and beta-lactalbumin, or
bovine, porcine, or human blood plasma comprising at least one of said proteins selected from the group consisting of albumin, transferrin, immunoglobulin G, immunoglobulin A, alpha-1-antitrypsin, and Factor VIII, or
egg white or egg yolk comprising at least one of said proteins selected from the group consisting of ovalbumin, lysozyme, immunoglobulin Y, ovomucoid, ovotransferrin, and avidin, or
plant tissue extracts, potato or legume extracts comprising at least one of said proteins selected from the group consisting of patatin, protease inhibitor, napin, cruciferin, legumin, and vicilin.

In a preferred embodiment, the biological composition is milk or whey.

The biological composition may also be separated as a mixture. The mixture would comprise the biological composition together with sorption material.

### Backward Flush System

In order to remove the dynamic layer of sorption material by end of the process or alternatively during the process if the layer for some reason becomes impermeable or show declined flow capacity, the system may be equipped with a backward flush system. Thus, in one embodiment, the apparatus further comprises an automatic backward flush system, such as an automatic backward flush system being configured to remove sorption material after elution of the target compound.

The backward flush system is applying a pulsed pressure increase and/or backward flush of the permeate. The increased pressure and/or backward flush of the permeate helps release any material that may have piled up, such as having created a flux reducing layer/film or having deposited in and on the membrane. Hence, in one embodiment, automatic backward flush system is configured to apply a pulsed pressure increase and/or backward flush of the permeate with a frequency in the range of 1-500 per hour and/or a pulse duration of between 0.01 to 200 seconds.

The backward flush system may be controlled by valve in the fluid connections. Thus, in one embodiment, the automatic backward flush system comprises an opening-closing valve (23) in the third fluid connection (17) being controlled by an electronic control unit (25). In a further embodiment, the automatic backward flush system comprises a first variable flow control valve (27) in the second fluid connection (13) and a second variable flow control valve (29) in the third fluid connection (17), that are being controlled by an electronic control unit (25).

The fluid used for the backward flush system may be permeate previously collected during the filtration process and stored in a permeate collection tank. Thus, in one embodiment the automatic backward flush system comprises permeate collection tank for collection of permeate via the third fluid connections (17) and/or the first outlet (18), a backward flush fluid connection connecting the permeate collection tank to the permeate side (9) of the membrane (5) for back-flushing of the membrane with permeate, and a centrifugal pump (35) inserted in the backward flush fluid connection and which centrifugal pump (35) is also controlled by the electronic control units (25).

The backward flush system is operating automatically and is preferably controlled by an electronic control unit (25). The frequency of operation may be very high with short durations for each operation performed. Thus, an embodiment of the present invention relates to the apparatus as described herein, wherein the pulsed pressure increase and/or backward flush of the permeate has a pulse duration of between 0.05 to 150 seconds.

Yet another embodiment of the present invention relates to the apparatus as described herein, wherein the pulse duration is in the range of 0.05 to 150 seconds, such as in the range of 0.1 to 120 seconds, such as in the range of 0.2 to 100 seconds, such as in the range of 0.3 to 80 seconds, such as in the range of 0.3 to 60 seconds, such as in the range of 0.4 to 50 seconds, such as in the range of 0.5 to 40 seconds, such as in the range of 0.5 to 30 seconds, such as in the range of 0.5 to 25 seconds, such as in the range of 0.5 to 20 seconds, such as in the range of 0.5 to 15 seconds, such as in the range of 0.5 to 10 seconds, such as in the range of 0.5 to 8 seconds, such as in the range of 0.5 to 6 seconds, such as in the range of 0.5 to 5 seconds, such as in the range of 1.0 to 5 seconds.

### Membrane

### Membrane Pore Size

The pore size of the membrane would depend on the sorption material to be used as the pore size of the membrane should allow a dynamic layer of sorption material to be formed. In one embodiment, the membrane has a nominal pore size in the range of 1 to 1000 μm, such as 1 to 950 μm, such as 5 to 800 μm, such as 10 to 600 μm, such as 20 to 500 μm, such as 30 to 300 μm, such as 50 to 200 μm. In yet an embodiment the membrane has a nominal pore size in the range of 2 to 50 μm, such as in the range of 5 to 40 μm, such as in the range of 10 to 30 μm

### Filtration System

An embodiment of the present invention relates to the apparatus as described herein, wherein the tangential flow filtration unit is a tubular filtration system or a cassette filtration system. The tangential flow filtration unit should be equipped with a membrane providing a high flux of permeate. The membrane may therefore be classified as a high flux filter.

Thus, an embodiment of the present application relates to the apparatus as described herein, wherein the membrane is a high flux filter with a clean water permeate flux of more than 1000 L/m²/h when the TMP is about 0.1-0.9 bar. More specifically, an embodiment of the present application relates to the membrane being a high flux filter with a clean water permeate flux of more than 1000L/m²/h, such as more than 2000 L/m²/h, such as more than 3000 L/m²/h, such as more than 4000 L/m²/h, such as more than 6000 L/m²/h at a retentate pressure of 0.5 bar. The numbers are shown in terms of the amount of clean water (L) passing through one square meter of the membrane (m²) per hour.

The tangential flow filter may be made of any material suitable for a filtration device to be applied in aqueous solutions. Thus, an embodiment of the present invention relates to the apparatus as described herein, wherein the membrane is of a filter material selected from the group consisting of porous inorganic material, porous organic polymer, and mixtures thereof.

A membrane according to the present invention could have been prepared by a method suitable for creating high flux filters, such as a method for creating high porosity filters. A particular embodiment of the present invention therefore relates to the filter material having been melt blown, spun, woven, sintered or cast to create the target porosity of the membrane.

### Membrane and Filter Device

The membrane is of a porous inorganic material and/or porous organic polymer material. A specific embodiment of the present invention therefore relates to the membrane being of a filter material selected from the group consisting of sintered metal, sintered metal powder, sintered metal strings and layers hereof, sintered metal oxides, sintered ceramics, porous glass, polypropylene, polyethylene, cellulose, cellulose acetate, viscose, polysulfone (PSU), polyethersulfone (PES), polyarylethersulfone (PAES), polyvinylidinedifluoride (PVDF), polytetraflouorethylene (PTFE), polyamide, nylon, polyester, surface modified PES, surface modified PTFE, and mixtures thereof.

An embodiment of the present invention relates to the filter material being porous sintered stainless steel particles or porous sintered stainless steel mesh.

A further embodiment of the present invention relates to the membrane being of a porous organic polymer material selected from the group consisting of polypropylene, polyethylene, cellulose, cellulose acetate, viscose, polysulfone (PSU), polyethersulfone (PES), polyarylethersulfone (PAES), polyvinylidinedifluoride (PVDF), polytetraflouorethylene (PTFE), polyamide, nylon, polyester, surface modified PES, surface modified PTFE, and mixtures thereof.

A further particular embodiment of the present invention relates to the membrane being a polypropylene filter. Another particular embodiment of the present invention relates to the membrane being a polyethylene filter. Yet another particular embodiment of the present invention relates to the membrane being a polyamide filter, such as a nylon filter. Just another particular embodiment of the present invention relates to the membrane being a melt blown polypropylene filter.

The membranes according to the present invention may have a variety of shapes and forms. Tangential flow filtration devices may be in the form of tubular or cylindrical, and they may be in the form of flat sheets, discs or plates. Flat sheet tangential flow filtration membranes may be spiral wound or they may be stacked with spacers in between each membrane layer. Also, the membrane and the filtration device may be designed for hygienic operations enabling efficient cleaning of the system before and after operation.

An embodiment of the present invention relates to the membrane being a tubular or cylindrical, or flat sheet filter.

In a particular embodiment of the present invention relating to the membrane being selected from the group consisting of cylindrical membrane, flat membrane, spiral wound membrane and tubular membrane.

When the membrane is a tubular filter or cylindrical filter, the inner diameter of the filter should be in the range of 15-1000 mm. An embodiment of the present invention therefore relates to the inner diameter of the cylindrical membrane or tubular membrane being in the range of 15-1000 mm, such as 15-750 mm, such as 15-500 mm, such as 15-400 mm, such as 15-300 mm, such as 25-500 mm, such as 25-400 mm, such as 25-300 mm, such as 40-500 mm, such as 40-400mm, such as 60-400 mm.

### Add-Ons

The apparatus of the present invention may be constructed to comprise additional parts, for example: mixing tanks, inlets, outlets, permeate collection tanks, valves, and pumps, etc.

The apparatus may comprise a mixing tank being fluidly connected to the tangential flow filtration unit via the first fluid connection. Via the first intlet biological composition and the sorption material may be mixed in the mixing tank prior to entering the tangential flow filtration unit. Hence, in one embodiment, the apparatus comprises one or more mixing tanks for containing a biological composition comprising a target compound mixed with the sorption material, a composition comprising sorption material and/or a biological composition comprising target compound, being fluidly connected to the tangential flow filtration unit. A mixing tank is illustrated in FIG. 3.

Yet another embodiment of the present invention relates to the apparatus as described herein, wherein the apparatus comprises one or more first inlets or outlets. Still another embodiment of the present invention relates to the apparatus as described herein, wherein the apparatus comprises one or more permeate collection tanks.

An embodiment of the present invention relates to the apparatus as described herein, wherein the second fluid connection is equipped with a heat exchanger. The heat exchanger makes it possible adjusting and controlling the temperature in the circulating flow. Control of the temperature may be important for obtaining a good viscosity of the mixture and/or an increased attachment of the target compound to the sorption material.

The third fluid connection is in some embodiments of the present invention e.g. as shown in FIG. 5, directed by further valves such that permeate is returned to a mixing tank or to the first inlet through a fourth fluid connection.

### Washing/Elution/Regeneration

### Washing

An embodiment of the present invention relates to the apparatus as described herein, wherein the apparatus comprises one or more inlets for adding a washing liquid. In one embodiment, the washing liquid is added via the first inlet. Alternatively or in addition, the apparatus may comprise washing tanks for easy addition of the washing liquid and for reducing risk of contamination. The washing tanks would be fluidly connected to the tangential flow filtration unit and the washing liquid may be drained via the first outlet or via separate outlet(s). Thus, an embodiment of the present invention relates to the apparatus as described herein, further comprising one or more washing tanks for containing a washing liquid and being fluidly connected to the tangential flow filtration unit.

The washing liquid is used for removing potential contaminants from the target compound and/or dynamic layer prior to eluting the target compound. In one embodiment, the washing liquid is water or an aqueous solution and/or an aqueous buffer solution. In a further embodiment, the washing liquid comprises an inorganic and/or organic acid and/or a salt hereof, such as an acid and/or a salt hereof comprising sodium or potassium. In yet a further embodiment, the washing liquid comprises one or more acids and/or salts hereof selected from the group consisting of hydrochloric acid, phosphoric acid, sulphuric acid, acetic acid, citric acid, caprylic acid, lactic acid and combinations thereof.

Additionally, the washing liquid may contain a detergent. Hence, in a further embodiment, the washing liquid comprises a detergent, such as sodium dodecyl sulfate.

Additionally, the washing liquid may contain an organic solvent soluble in water. Hence, in a further embodiment, the washing liquid comprises an organic solvent soluble in water, such as ethanol, propylene glycol and acetone.

Furthermore, it is important that the purified target compound may be used for the purposes wished for. Thus, if the target compound is to be used for food or feed, it is important that the washing liquid is food grade. Hence, in one embodiment, the washing liquid is food grade.

### Elution

Elution of the target compound from the sorption material may be performed by adding an elution liquid by use of a system installed for the purpose. An embodiment of the present invention therefore relates to the apparatus as described herein, wherein the apparatus comprises one or more inlets for adding an elution liquid. In one embodiment, the elution liquid is added via the first inlet. Alternatively or in addition, the apparatus may comprise elution tanks for easy addition of the elution liquid and for reducing risk of contamination. Thus, a further embodiment of the present invention relates to the apparatus as described herein, wherein the apparatus comprises one or more elution tanks for containing an elution liquid and being fluidly connected to the tangential flow filtration unit. The elution tanks would be fluidly connected to the tangential flow filtration unit and the elution liquid may be drained via the first outlet or via separate outlet(s).

The elution liquid is used for eluting the target compound from the sorption material. In one embodiment, the elution liquid is water or an aqueous solution and/or an aqueous buffer solution. In a further embodiment, the elution liquid comprises an inorganic and/or organic acid and/or salts hereof, such as an acid salt comprising sodium or potassium. In yet another embodiment, the elution liquid comprises one or more acids and/or salts hereof selected from the group consisting of hydrochloric acid, phosphoric acid, sulphuric acid, acetic acid, citric acid, caprylic acid, lactic acid and combinations thereof.

Additionally, the washing liquid may contain a detergent. Hence, in one embodiment, the elution liquid comprises a detergent, such as sodium dodecyl sulfate.

Additionally, the elution liquid may contain an organic solvent soluble in water. Hence, in a further embodiment, the washing liquid comprises an organic solvent soluble in water, such as ethanol, propylene glycol and acetone.

Furthermore, it is important that the purified target compound may be used for the purposes wished for. Thus, if the target compound is to be used for food or feed, it is important that the washing liquid is food grade. Hence, in one embodiment, the elution liquid is food grade.

### Regeneration

The apparatus of the present invention is designed such that the sorption material may be used for a very long time without replacement or regeneration in an external regeneration system. Thus, the apparatus of the present invention may comprise regeneration tanks for easy addition of a regeneration liquid and for reducing any risk of contamination. An embodiment of the present invention therefore relates to the apparatus as described herein, wherein the apparatus comprises one or more regeneration tanks for containing a regeneration liquid and being fluidly connected to the tangential flow filtration unit. In another embodiment of the present invention relating to the apparatus as described herein, the apparatus comprises one or more inlets for adding a regeneration liquid. In one embodiment, the elution liquid is added via the first inlet. During or after regeneration of the sorption material the regeneration liquid may be drained by use of outlets installed for the purpose. The elution tanks would be fluidly connected to the tangential flow filtration unit and the elution liquid may be drained via the first outlet or via separate outlet(s).

Without being restricted to any particular liquids for use in regeneration of the sorption material, a particular embodiment of the present invention relates to the apparatus as described herein, wherein the regeneration liquid is a basic solution having a pH-value in the range of 8 to 14, such as 10 to 14, such as 12 to 13.

It is to be understood that the first inlet and the inlets above as well as the first outlet and the outlets described above may be the same inlets or outlets. Alternatively, they are individual inlet or outlets.

### Pumps

The apparatus according to the present invention may comprise several pumps such as a circulation pump, a permeate pump and a back flush pump.

Advantageously, these pumps are gentle towards the compounds being separated and the sorption material, whereby the same adsorbent material may be employed progressively and stay as an integral part of the apparatus. Thus, in one embodiment the pumps are positive displacement pumps. Another embodiment of the present invention relates to the apparatus as described herein, wherein the positive displacement pump is a low-shear pump. Further in this regard, the pumps may be single rotor low-shear pumps selected from the group of vane pump, piston pump, progressing cavity pump, screw pump, and peristaltic pump; or selected from the group of multiple rotor low-shear pumps comprising gear pump, lobe pump, circumferential piston pump, and screw pump. An embodiment of the present invention therefore relates to the apparatus as described herein, wherein the positive displacement pump is selected from the group consisting of vane pump, piston pump, progressing cavity pump, screw pump, gear pump, lobe pump, circumferential piston pump, screw pump, and peristaltic pump, preferably peristaltic pump. Alternatively the circulation pump may be a low-shear centrifugal pump.

The low shear force exerted by the pumps, said fluid connections and other parts, increases the life-time of the sorption material. Use of the apparatus according to the present invention is therefore considered to be particularly cost-efficient and environmentally friendly. By low shear force is to be understood that the same sorption material is circulated around in the fluid connections and parts between the mixing tank and the retentate side of the filter, for at least 50 rounds, such as at least 500 rounds, such as at least 1000 rounds, such as at least 5000 rounds, such as at least 10.000 rounds and/or that each particle of sorption material may adsorb a compound molecule more than 50 times, such as 500 times, such as 1000 times, such as 5000 times, such as 10.000 times without being degraded i.e. at least 90% of the sorption material is within the size range of the sorption material from a start.

### The Process

A further aspect of the present invention relates to a process for separation of a target compound comprised in a biological composition, the process comprising the steps of:
i) adding to a tangential flow filtration unit comprising a membrane having a retentate site and a permeate site:
   a composition comprising a sorption material capable of adsorbing a target compound, or
   a mixture comprising a sorption material mixed with a biological composition comprising a target compound, whereby the target compound is adsorbed by the sorption material;
ii) filtrating the composition or the mixture to obtain a retentate comprising the sorption material, optionally with adsorbed target compound, and a permeate,
iii) recirculating at least part of the retentate on the tangential flow filtration unit, gradually forming a dynamic layer of said sorption material, optionally with adsorbed target compound, on the retentate site of the membrane during the recirculation of the retentate,
iv) optionally, adding a biological composition comprising target compound, filtrating the biological composition through the dynamic layer of sorption material and recirculating the retentate, whereby the target compound is adsorbed by the sorption material;
v) adding an elution liquid to release the target compound from the sorption material, and filtrating the retentate using the tangential flow filtration unit to obtain an eluted retentate comprising the sorption material and a target-containing permeate comprising the target compound;
vi) optionally, collecting the target-containing permeate and/or the permeate of steps ii)-iv) as separated product composition(s).

In a particular embodiment, the present invention relates to the tangential fluid flow being a continuous tangential fluid flow.

In a particular embodiment of the present invention, the sorption material is initially added as a composition such as a suspension in water or other suitable aqueous solution before the biological composition is added. In this case the biological composition comprising the target compound may then be added (feeded) gradually by a feeding pump while the sorption material is recirculated through the tangential flow filtration unit. In one embodiment, the permeate is taken out (bleeded) with a flow rate which is substantially equal to the feeding flow rate of the biological composition.

During the process the biological composition or the mixture may be added to the tangential flow filtration unit in a batch-wise manner i.e. in one instance. Alternatively, the biological composition or the mixture may be added continuously, whereby it will be mixed with the recirculated retentate in a continuously manner. This is particularly advantageous when purifying large quantities of target compounds. Hence, in one embodiment, the biological composition or mixture is added continuously.

Similarly, washing and elution steps may be performed by a continuous and simultaneous feed and bleed.

However, for some applications, one equilibrium stage may be enough for the specific purpose and in this case the mixing of the sorption material and the biological composition may be performed in one instant mixing step, while the subsequent washing and elution steps is performed as continuous and simultaneous feed and bleed.

To obtain proper binding of target compound to the sorption material in the mixture, the mixture is preferably recirculated prior to filtration. Thus, in a further embodiment, the process comprises a further step of recirculating the mixture over the tangential flow filtration unit prior to step ii). In a further embodiment, the permeate pump is disengaged during this additional step.

The volumetric fluid flows (measured e.g. as L/min) of the permeate and the retentate may be adjusted in order to adjust the dynamic layer of the sorption material. This adjustment is particularly advantageous in step iii). However, the fluid flows may also be relatively adjusted in any of the other steps. A particular embodiment of the present invention relates to the fluid flows of the retentate and the permeate in step iii) are adjusted such that the permeate flow is at least 5%, such as at least 10%, such as at least 15%, such as at least 20%, such as at least 25%, such as at least 30%, such as at least 35%, such as at least 40%, such as at least 45%, such as at least 50% of the retentate flow entering the tangential flow filtration unit; or in a range of 5-90%, such as 10-80%, such as 20-70%, such as 25-65%, such as 30-60% of the retentate flow entering the tangential flow filtration unit.

In one embodiment, the linear fluid flow of the retentates in the range of 1-200 cm/s, such as in the range of 1-150 cm/s, such as in the range of 1-100 cm/s, such as in the range of 1-75 cm/s, such as 1-50 cm/s, such as in the range of 1-40 cm/s, such as in the range of 1-30 cm/s, such as in the range of 2-80 cm/s, such as in the range of 2-60 cm/s, such as in the range of 2-40 cm/s. The linear flow rate of the retentate may change during the process due to the adsorbent layer being formed, even though the volumetric flow rate may be kept constant.

In a further embodiment, the per minute volumetric fluid flow rate of the permeate is in the range of 0.1 to 10 times the volume of sorption material in the apparatus, such as 0.2 to 8, such as 0.3 to 6, such as 0.4 to 5, such as 0.5 to 4, such as 0.5 to 3, such as 0.5 to 2 times the volume of sorption material in the apparatus.

In one embodiment, the membrane has a pore size of 1-1000 μm and a permeate flow being 30-60% of the retentate flow entering the tangential flow filtration unit. In a further embodiment, the membrane has a pore size of 2-50 μm and a permeate flow being 30-60% of the retentate flow entering the tangential flow filtration unit.

The fluid flows of the permeate and the retentate is adjusted to obtain a particular thickness of the dynamic layer. Thus, in one embodiment, the process further relates to the dynamic layer of the sorption material reaching a thickness of at least 1 mm, such as at least 3 mm, such as at least 5 mm, such as at least 10 mm, such as at least 15 mm, such as at least 20 mm, such as at least 25 mm, such as at least 30 mm, such as at least 35 mm, such as at least 40.

In order for the fluid flow through the dynamic layer of sorption material and the membrane to be evenly distributed, a partial vacuum is advantageously formed at the permeate site of the membrane e.g. by use of a valve arranged in the tangential flow filtration unit. Thus, in one embodiment, the process further comprises a step of introducing a partial vacuum on the permeate side of the membrane. In a further embodiment, the partial vacuum results in a pressure on the permeate side in the range of 0.10 to 0.98 bar, such as in the range of 0.20 to 0.95 bar, such as in the range of 0.30 to 0.95 bar, such as in the range of 0.50 to 0.95 bar, such as in the range of 0.60 to 0.95 bar, such as in the range of 0.70 to 0.95 bar, such as in the range of 0.75 to 0.90 bar.

Surprisingly, it has been observed that if the partial vacuum is combined with a substantial drainage of liquid from the permeate side of the membrane, this results in an even more evenly distributed fluid flow. Thus, in a further embodiment, the present invention relates to introducing a partial vacuum on the permeate side of the membrane together with a drainage of liquid from the permeate side of the membrane whereby a substantial drainage of liquid is obtained. By substantial drainage is understood that at least 50%, such as at least 60%, such as at least 70%, such as at least 75%, such as at least 80%, such as at least 85%, such as at least 90%, such as at least 95% of the permeate compartment volume (41) on the permeate side of the tangential flow filtration unit is filled with air.

According to the process according to the present invention, the retentate is at least partly circulated. In one embodiment, all of the retentate except the sorption material maintained in the dynamic layer of sorption material is recirculated.

The recirculation of the retentate is performed in order for sufficient of the target compound to be bound to the sorption material. In one embodiment, the retentate is recirculated over said tangential flow filtration unit until said sorption material has adsorbed at least 1 g/L, such as at least 5 g/L, such as at least 10 g/L, such as at least 15 g/L, such as at least 20 g/L, such as at least 25 g/L, such as at least 30 g/L, such as at least 35 g/L, such as at least 40 g/L of said target compound. The amount of target compound adsorbed is measured by e.g. spectrometry.

In addition to recirculating the retentate also the eluted retentate may be recirculated until the target compound has been released from the sorption material, such as sufficiently released, like at least 90% of bound target compound in the sorption material has been released from the sorption material. Thus, in one embodiment the eluted retentate is recirculated over said tangential flow filtration unit until said target compound has been released from said sorption material.

To further improve the separation process, the process may comprise further steps in the process. In one embodiment, the process also comprises one or more washing step(s) prior to adding the elution liquid, preferably between step iii) and step v) or step iv) and step v), said washing step(s) comprises adding a washing liquid to the retentate and following filtrating using said tangential flow filtration unit to obtain a washed retentate and a washing permeate. In a further embodiment, the washed retentate is recirculated over said tangential flow filtration unit.

In addition, the process may comprise a step at the end of the process to regenerate the sorption material. Thus, in one embodiment, the process comprises a further step of adding a regeneration liquid after said separated product composition has been collected, to regenerate the properties of said sorption material.

The mixture added in the process comprises sorption material mixed with a biological composition comprising a target compound, where the target compound is adsorbed by the sorption material prior to adding it to the tangential flow filtration unit. In one embodiment, the mixture comprises a sorption material in an amount of at least 0.1% by volume, such as at least 1% by volume, such as at least 5% by volume, such as at least 7% by volume such as at least 10% by volume, such as at least 15% by volume, such as at least 20% by volume, such as at least 25% by volume, such as at least 30% by volume, such as at least 35% by volume, such as at least 40% by volume, such as in the range of 5-55% by volume, such as in the range of 10-50% by volume, such as in the range of 15-45% by volume, such as in the range of 20 to 40 percentage by volume based on the total volume of the mixture.

In one embodiment, the membrane has a pore size of 1-1000 μm, a permeate flow being 30-60% of the retentate flow entering the tangential flow filtration unit and the mixture comprises a sorption material in the range of 20-40% by volume based on the total volume of the mixture.

In a further embodiment, the membrane has a pore size of 2-50 μm, a permeate flow being 30-60% of the retentate flow entering the tangential flow filtration unit and the mixture comprises a sorption material in the range of 20-40% by volume based on the total volume of the mixture.

In one embodiment, the membrane has a pore size of 1-1000 μm, a permeate flow being 30-60% of the retentate flow entering the tangential flow filtration unit, the mixture comprises a sorption material in the range of 20-40% by volume based on the total volume of the mixture and the sorption material has a size distribution such that 90% by volume of the particles have a diameter in the range of 30-250 μm.

In a further embodiment, the membrane has a pore size of 2-50 μm, a permeate flow being 30-60% of the retentate flow entering the tangential flow filtration unit, the mixture comprises a sorption material in the range of 20-40% by volume based on the total volume of the mixture and the sorption material has a size distribution such that 90% by volume of the particles have a diameter in the range of 30-250 μm.

In one embodiment, the membrane has a pore size of 1-1000 μm, a permeate flow being 30-60% of the retentate flow entering the tangential flow filtration unit, the mixture comprises a sorption material in the range of 20-40% by volume based on the total volume of the mixture and the sorption material is spherical, with a particle diameter in the range of 10-60 μm, and a density in the range of 1.01 to 1.10 g/ml.

In a further embodiment, the membrane has a pore size of 2-50 μm, a permeate flow being 30-60% of the retentate flow entering the tangential flow filtration unit, the mixture comprises a sorption material in the range of 20-40% by volume based on the total volume of the mixture and the sorption material is spherical, with a particle diameter in the range of 10-60 μm and a density in the range of 1.01 to 1.10 g/ml.

In one embodiment, the membrane has a pore size of 1-1000 μm, a permeate flow being 30-60% of the retentate flow entering the tangential flow filtration unit, the mixture comprises a sorption material in the range of 20-40% by volume based on the total volume of the mixture and the sorption material is spherical, with a particle diameter in the range of 30-300 μm and a density in the range of 1.01 to 1.10 g/ml.

In a further embodiment, the membrane has a pore size of 2-50 μm, a permeate flow being 30-60% of the retentate flow entering the tangential flow filtration unit, the mixture comprises a sorption material in the range of 20-40% by volume based on the total volume of the mixture and the sorption material is spherical, with a particle diameter in the range of 30-300 μm and a density in the range of 1.01 to 1.10 g/ml.

The process may additionally comprise a further step of applying a backward flush in order to loosen the adsorption layer fully or partly from the retentate side of the membrane. Whether it is removed fully or partly would depend on the intensity of the back flush procedure. The desired effect may be to decrease the flow resistance of the adsorbent layer caused by compression and/or build-up suspended particles present in the raw material. Thus, in one embodiment the process further comprises a step of applying a backward flush to said tangential flow filtration unit, such as after step vi).

The backward flush may be obtained by amending the pressure difference between the retentate side and the permeate side of the tangential flow filtration unit e.g. by means of valves and pumps present in the apparatus. Hence, in one embodiment, a pressure difference between the retentate side and permeate side of the membrane in the tangential flow filtration unit is equalized or reversed resulting in a pulsed pressure increase and/or backward flush of the permeate or any desired alternative back flush liquid is obtained, said pressure difference is introduced at least once during the process during one or more of the steps ii) to v).

The permeate throughout the process may comprise compounds of interest if the target compound is e.g. an impurity. If however, the target compound is the compound of interest only the permeate of the last step i.e. the target-containing permeate is of interest, while the remaining permeate may in some embodiments be considered as a waste product. However, this waste product may be collected for different purposes such as for use in applying a backward flush on the tangential flow filtration unit. In another embodiment, both the permeate containing the target compound and the permeate from which the target compound has been removed are of interest.

Accordingly, the permeate, whether comprising the target compound or not, may be a permeate of value for different purposes, why the collection of permeate at different stages of the process would be of advantage. Thus, in one embodiment, the permeate is collected in a waste tank or a permeate collection tank.

In a particular embodiment the apparatus according to the invention is employed for so-called “single-use” adsorbent procedures wherein the adsorbent is only used for one cycle of adsorption, or only a few cycles, such as 2 cycles, such as 4 cycles, such as 6 cycles, such as 8 cycles, such as 10 cycles, such as 15 cycles of adsorption, to process a specific batch of raw material, such as fermentation broths from mammalian cell cultures to produce biopharmaceuticals.

It should be noted that embodiments and features described in the context of one of the aspects of the present invention also apply to the other aspects of the invention.

All patent and non-patent references cited in the present application, are hereby incorporated by reference in their entirety.

The invention will now be described in further details in the following non-limiting examples.

### EXAMPLES

FIG. 5 shows a schematic representation of an apparatus used for the examples 1 and 3.

The chemicals used in the examples, e.g. for preparing buffers and solutions are commercial products of at least reagent grade. Water used for conducting the experiments was de-ionized water.

All tests were performed at room temperature unless otherwise stated.

### Example 1—Test of Apparatus and Forming an Adsorbent Layer Inside Filter Unit

The mixing tank (37) having a first inlet (1) was a cylindrical stainless steel tank with a conical bottom. The first fluid connection (11) comprised a variable flow peristaltic tube pump (15) having a maximum flow capacity of 10 L/min. The tangential flow filtration unit (3) having a feed entry and a retentate exit was a cylindrical unit equipped with a filter membrane (5) in the form of a filter tube made of melt blown polypropylene and having a nominal pore size of 30 micron.

The filter tube had a length of 300 mm, an outer diameter of 76 mm and an inner diameter of 66 mm. The total inner surface filter area of the filter tube was 0.062 m² and the inner volume of the filter was 1.03 L. The retentate exit was connected to the second fluid connection (13) leading the retentate back to the mixing tank (37). The permeate produced during the filtration process was retrieved through a third fluid connection (17), which was connected to a permeate outlet pump (19) and a two-way valve (39) leading permeate back to the mixing tank through the fourth fluid connection (33) or to a permeate collection tank via the first outlet (18).

For testing of the apparatus with adsorbent the mixing tank was filled with 3.2 L of an aqueous suspension of 6% cross-linked agarose beads with a particle size in the range of 45-165 micron (Cytiva, USA). The concentration of cross-linked agarose beads in the suspension was 30%, as determined by centrifugation of a sample of the suspension for 5 minutes at 3000 G (i.e. a 100 ml sample would have 30 ml sedimented beads after centrifugation). The system volume at the outset of the test was thus 3.2 L and the total volume of adsorbent beads was 0.96 L.

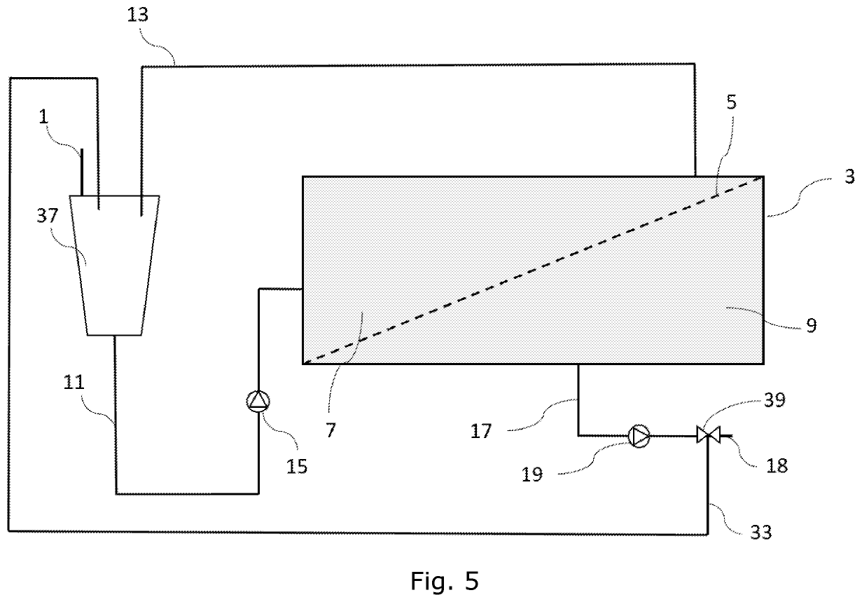

In the first phase of the test, the retentate circulation pump was set to a flow rate of 4.0 L/min while the permeate pump remained unengaged (i.e. no permeate flow was established). This volumetric flow rate corresponds to a linear flow rate inside the filter of approx. 117 cm/min (1.9 cm/s). After a few minutes upstart the permeate pump was engaged to give a permeate flow of 0.2 L/min. This flowrate corresponds to a permeate flux of (0.2×60 L/min)/0.062 m2=194 L/min×m2 (LMH). The permeate was recycled to the mixing tank to keep the system volume constant. After 10 minutes of recirculation, a sample of 10 ml was collected from the mixing tank and centrifuged to determine the concentration of adsorbent beads. Following this, and while keeping the flow rate of the retentate circulation pump constant at 4.0 L/min, the permeate flowrate was increased in steps (FIG. 6) and for each step a 10 ml sample was collected from the retentate tank and centrifuged to determine the concentration of adsorbents beads. After centrifugation and read out of the concentration of beads each sample was returned to the mixing tank to keep the system volume constant during the test.

At the end of the first test, the layer of adsorbent beads formed on the inside of the filter tube was resuspended by reversing the permeate pump and pumping in water in the opposite direction from the permeate to the retentate side (backward flushing). When the adsorbent layer was completely removed (as determined by measuring the concentration of adsorbent in the retentate) the permeate pump was again reversed to slowly remove the water injected to the retentate during the back flush.

Following this, the stepwise permeate flow procedure was repeated twice using a fixed flow rate of the retentate circulation pump of 2 L/min and 8 L/min, respectively.

### Results

From the results obtained, the concentration of adsorbent beads in the retentate was used to calculate the fraction of the total amount of adsorbent retained as a layer inside the filter tube as a function of the permeate flow rate relative to the flow rate of the retentate circulation pump, see FIG. 6.

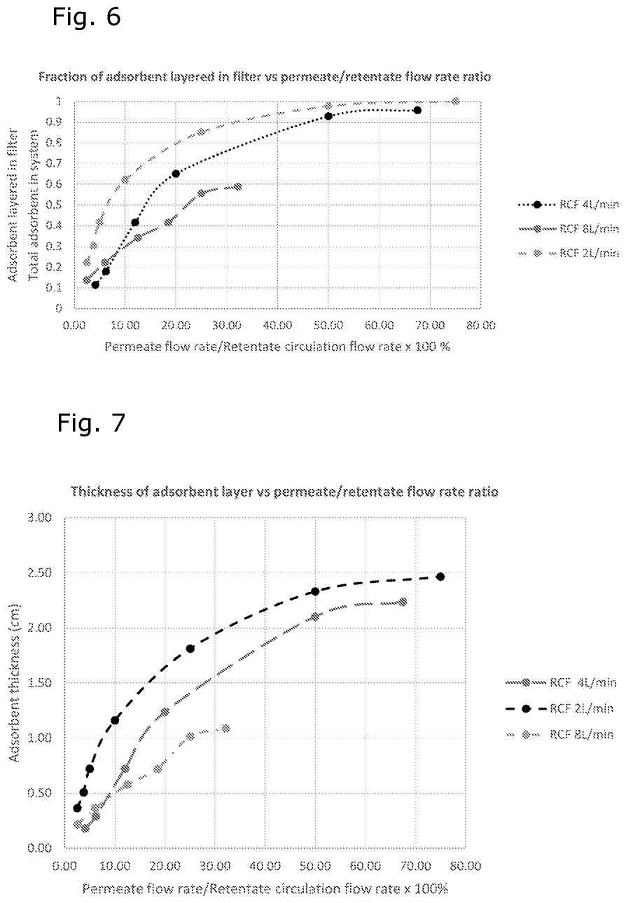

Also, from the same data, it was calculated how the average thickness of the adsorbent layer inside the filter tube varied as a function of the permeate flow rate relative to the flowrate of the retentate circulation pump, see FIG. 7.

The results illustrate that the control of the permeate flow rate relative to the retentate circulation flow rate may be used to control the formation of an adsorbent layer inside the filter tube of the cross flow filtration unit.

As indicated in FIG. 6, the fraction of total amount of adsorbent layered inside the filter tube strongly depends on the permeate flow rate relatively to the retentate circulation flow rate. At low relative permeate flow rate, e.g. at about 3-5% of the retentate circulation flow rate only about 10-20% of the adsorbent volume is layered inside the filter tube, while at higher relative permeate flow rate, e.g. about 40-50% of the retentate circulation flow rate between 80-100% of the adsorbent volume may be layered inside the filter tube.

Likewise, FIG. 7 illustrates the average thickness of the adsorbent layer building up as a function of the relative permeate flow rate, being approx. 2.5 cm under the conditions where the total amount of adsorbent is being layered inside the filter tube.

### Conclusion

Accordingly these data demonstrate how the relationship between the permeate flow and the retentate flow can control the amount of sorption material absorbed to the retentate site of the filter as well as the average thickness of the layer.

### Example 2—Preparation of a Quaternary Amine Ion Exchange Adsorbent

A quaternary amine ion exchange adsorbent was prepared by cross-linking of 6% agarose beads having a particle size range of 45-160 micron (Cytiva, USA). The beads were reacted with epichlorohydrin as follows: 20 L beads were suspended in 15 L 0.2 M sodium sulphate (Sigma Aldrich, USA) in a closed reaction vessel with stirring. The suspension was heated to 40° C. and then added 1.2 L epichlorohydrin (Sigma-Aldrich, USA) and 1.5 L 32.5% (w/w) sodium hydroxide. The suspension was stirred and maintained at 40° C. for 18 hours and then it was transferred to a nutsche filter and washed with 200 L water. The beads were then drained for interstitial water whereby the cross-linked beads could be removed as a filter cake.

Reaction with quaternary amine reagent: The cross-linked beads were then suspended in 15 L 0.6 M sodium hydroxide and transferred back to the reaction vessel as a suspension. Following, the suspension was added 994 g sodium sulphate and heated to 65° C. under gentle stirring. Hereafter, 12 kg glycidyl-trimethyl-ammonium chloride (Sigma Aldrich, USA) was added under continuous stirring in 10×1 kg aliquots with 30 minutes intervals. The temperature was regulated to remain within the range of 55-65° C. Following, the last addition of the reagent the mixture was allowed to react for an additional 20 hours. After the reaction the suspension was transferred to a nutsche filter and washed sequentially with 200 L water, 200 L 0.05 M hydrochloric acid and 200 L water. The beads were then drained for interstitial water whereby the quaternary amine coupled beads could be removed as a filter cake.

### Results & Conclusion

The washed and drained beads were found to have a content of 175 micromole quaternary amine groups per gram drained beads.

### Example 3—Isolation of Beta-Lactoglobulin from Bovine Skim Milk

This example demonstrates the use of the apparatus according to the invention, such as that illustrated in FIG. 5, for isolation of beta-lactoglobulin (beta-lg) selectively from bovine skim milk.

### Materials and Methods

### Adsorbent:

Quaternary amine ion exchange adsorbent, prepared as described in example 2.

### Raw Material:

Skim milk was sourced from a local dairy.

### Buffer Solutions

0.1 M sodium citrate pH 4.0
19,2 g of citric acid, from BDH Chemicals
Addition of 900 ml of water and pH is adjusted to 4.0 with 1 M NaOH. Water is then added to a final volume of 1.0 L.

### Equipment Setup

The tangential flow unit is shown in FIG. 8

### SDS-Page Electrophoresis Reagents

a) LDS sample buffer, 4X is obtained from Abcam, UK
b) SDS Run buffer, 20x is obtained from Abcam, UK
c) Precast 16% gels are obtained from Abcam, UK
d) Instant Stain 15 min Coomassie blue for proteins was obtained from Kem-En-Tec Nordic, DK.

### SDS-Page Electrophoresis

The samples produced in each example were analysed using SDS-PAGE gel electrophoresis showing the protein composition in each sample. The SDS-PAGE gel electrophoresis was performed using an electrophoresis apparatus and precast 16 gels from Abcam, UK. The protein samples were mixed with LDS sample buffer and incubated for 10 minutes at 70° C. The samples were applied to the precast gel and proteins were allowed to run for one hour at 200 V 90 mA in the SDS Run buffer at non-reduced running conditions. The gel was developed in the staining solution for 18 hours. The protein bands were evaluated by visually inspection or analysed by scanning densitometry to quantify the amount of specific proteins in the test solutions.

### Tangential Flow Unit

The tangential flow filtration unit (3) was a cylindrical unit equipped with a filter tube (5) made of melt blown polypropylene having nominal pore size of 30 micron. The dimensions of the filter tube were: outer diameter: 76 mm, inner diameter: 66 mm, and length: 300 mm. The filtration area in the filter tube was calculated to be: 0.062 m² and the volume of the filter tube was calculated to be 1.03 L.

The filtration unit was further equipped with a valve (21) for inlet of air to the permeate side (9) of the filter (5) in the tangential flow filtration unit (3) i.e. outside of the filter tube and the inside of the filter house (43).

### Procedure-Batch Adsorption Followed by Wash and Elution in Tangential Flow Filter

8 L skim milk and 0.9 L quaternary amine ion exchange adsorbent was added to the mixing tank of the apparatus and the peristaltic tube pump was set to recirculate the mixture through the tangential flow filtration unit at a flow rate of 4 L/min with the permeate pump disengaged (i.e. no permeate flow). In this way the ion exchange adsorbent was kept in constant and well mixed suspension to ensure proper contact with all the skim milk in the mixture. Following recirculation for 30 minutes the permeate pump was engaged to give a permeate flow of 2.5 L/min. The permeate, free of adsorbent particles, was collected in a separate permeate tank and subsequently analysed by SDS-PAGE to confirm that the beta-lactoglobulin was efficiently bound to the ion exchanger.

When the mixing tank was close to empty (approx. 100 ml skim milk left in the tank) water was added. An external feed pump was pumping water into the mixing tank to wash out non-bound skim milk components at a flow rate of 2.5 L/min, thus keeping the system volume constant from this point. At initiation of the washing step the air inlet valve (21) was briefly opened such that the liquid present in the permeate compartment (41) was drained out and the compartment instead was filled with air. When the liquid level had reached 2 cm above the permeate outlet (45) the air inlet valve (21) was again closed such that a partial vacuum in the permeate compartment was created by the permeate pump. This procedure ensures an even flow of permeate throughout the entire surface area of the filter tube.

In total 10 L of water was used for the washing step. By the end of the washing step a sample of the retentate was withdrawn to confirm that practically all the ion exchange adsorbent was now layered inside the filter tube of the tangential flow filtration unit.

While now setting the peristaltic retentate circulation pump at a flow rate of 2 L/min and the permeate pump at 1 L/min elution of the bound beta-lactoglobulin was now initiated by using the external feed pump to pump in 100 mM sodium citrate pH 4.5 into the mixing tank at a flow rate of 1 L/min, thus still keeping the system volume constant and at a minimum level in the mixing tank. In total 10 L sodium citrate buffer was added to the mixing tank.

The elution permeate was collected in fractions of 0.5 L and the absorbance at 280 nm was determined to follow the progress of the elution and identify the fractions containing beta-lactoglobulin. SDS-PAGE electrophoresis was used to confirm that the eluted protein was highly purified beta-lactoglobulin.

Following complete elution of the bound beta-lactoglobulin and while now setting the peristaltic retentate circulation pump to a flow rate of 8 L/min, the tangential flow filtration unit was subsequently back-flushed by reversing the flow direction of the permeate pump and now feeding it with water at a flow rate of 4 L/min. Hereby the filter tube was washed with water from the outside to the inside and the anion exchange adsorbent was pushed away from the inner surface of the filter and resuspended in the circulating water into the mixing tank. Following this, the permeate pump was disengaged such that no permeate flow took place and the mixing tank was added an alkaline cleaning agent (pH 13) while keeping the anion exchanger in well mixed suspension by the constant recirculation of the retentate. By this procedure, it was ensured that the ion exchanger was efficiently cleaned and any suspended solids (such as milk fat globules) that might have been captured in the adsorbent layer was released and efficiently exposed to the cleaning agent for complete dissolution. Following two hours of cleaning without permeate flow the permeate pump was reengaged to give a permeate flow of 2L/min while the circulation pump was adjusted to 4 L/min. Hereby the ion exchange adsorbent was again forming a layer inside the filter tube. When the mixing tank was close to empty a further few litres of alkaline cleaning liquid was added by the external feeding pump to completely remove the first cleaning volume. Following this the external feeding pump was engaged to add water at a flow rate of 2 L/min and this washing procedure was continued until the pH of the permeate was below pH 8.0.

The apparatus was now ready for a next cycle of beta-lactoglobulin binding from skim milk. To initiate the next adsorption cycle the permeate pump would now again be reversed and the next batch of skim milk would be pumped into the apparatus from the outside to the inside of the filter tube. Hereby the ion exchange adsorbent would be resuspended in the mixing tank and the resulting mixture of skim milk and adsorbent could again be recirculated in the mixing tank for 30 minutes with the permeate pump disengaged, whereafter the procedure as described above would be repeated.

### Results

The collected elution permeate fractions were analysed by spectrophotometry (VWR, UV-3100PC) to determine the absorbance at 280 nm, and calculating the approximate beta-lactoglobulin concentration of each fraction by using the extinction coefficient:

$$OD - 280 nm ( 0.1 \% ) = 0.95 .$$

All fractions having a beta-lactoglobulin concentration at or above 1 g/L was hereafter pooled into one eluate fraction. The total volume of the eluate fraction was 3.0 L and the yield of beta-lactoglobulin in this fraction was 26.4 g.

SDS-PAGE analysis confirmed that practically all beta-lactoglobulin was adsorbed to the anion exchange adsorbent and that the purity of the subsequently eluted beta-lactoglobulin was above 90% relative to any other proteins in the pooled eluate fractions (data not shown).

### Conclusion

The results illustrate an important aspect of the invention in that the obtained pooled eluate fraction has a volume of only 3.0 L, which corresponds to approximately 3.3 times the volume of the ion exchange adsorbent used for the test. When running the same type of adsorption procedure under conditions wherein the adsorbent is kept in suspension in the mixing tank (high relative retentate circulation flow and higher system volume) the elution volume would be more than 100-200% higher than what is achieved in the apparatus and procedure according to the invention. This difference is highly significant in an industrial setting since the usage of water and the cost of concentrating the beta-lactoglobulin product would be considerably lower when using the apparatus and procedure according to the invention.

### DRAWING NUMBERS

1: First inlet
3: Tangential flow filtration unit
5: Membrane
7: Retentate site
9: Permeate site
11: First fluid connections
13: Second fluid connections
15: Circulation pump
17: Third fluid connections
18: First outlet
19: Permeate pump
21: Valve
23: Opening-closing valve
25: Electronic control unit
27: First variable flow control valve
29: Second variable flow control valve
31: Sorption particle
32: Thickness of the dynamic layer
33: Fourth fluid connection
35: Centrifugal pump
37: Mixing tank
39: Two-way valve
41: Permeate compartment
43: Filter house
45: Permeate outlet

1. An apparatus comprising
a first inlet 1;
a tangential flow filtration unit 3 comprising a membrane 5 having a retentate site 7 and a permeate site 9 and a nominal pore size in the range of 10 to 1000 μm;
one or more first fluid connections 11 leading fluid from said first inlet 1 to said tangential flow filtration unit 3;
one or more second fluid connections 13 leading retentate from said tangential flow filtration unit 3 to said first inlet 1 and/or said first fluid connections 11;
a circulation pump 15 for re-circulation of fluid through said tangential flow filtration unit 3 via said first and second fluid connections 11,13;
one or more third fluid connections 17 for leading permeate to a first outlet 18; and
an adsorbent,
wherein said adsorbent comprising chemically modified porous particles having an average particle size of at least 20 micron; and
said apparatus further comprises a permeate pump 19, said permeate pump 19 and the circulation pump 15 is adapted to, when in use, form a dynamic layer of said adsorbent on the retentate side 7 of said membrane 5 with a thickness of at least 1 mm, such as at least 3 mm, such as at least 5 mm, such as at least 10 mm, such as at least 15 mm, such as at least 20 mm, such as at least 25 mm, such as at least 30 mm, such as at least 35 mm, such as at least 40 mm.

2. The apparatus according to claim 1, wherein said permeate pump 19 is arranged in connection with the one or more third fluid connections 17.

3. The apparatus according to claim 1, wherein said apparatus further comprises a valve, wherein the valve 21 is capable of inlet or outlet of air to the permeate site 9 of the membrane 5, and optionally, wherein said valve 21 is arranged in connection with the tangential flow filtration unit 3.

4. The apparatus according to claim 1, wherein said sorption material is spherical.

5. A process for separation of a target compound comprised in a biological composition, the process comprising the steps of:
i) providing the apparatus according to claim 1;
ii) adding to a tangential flow filtration unit 3 comprising a membrane 5 having a retentate site 7 and a permeate site 9:
a) a composition comprising an adsorbent capable of adsorbing a target compound, or
b) a mixture comprising an adsorbent mixed with a biological composition comprising a target compound, whereby the target compound is adsorbed by adsorbent
wherein the adsorbent comprising chemically modified porous particles having an average particle size of at least 20 micron;
iii) filtrating the composition or the mixture to obtain a retentate comprising the adsorbent, optionally with adsorbed target compound, and a permeate,
iv) recirculating at least part of the retentate on the tangential flow filtration unit, forming a dynamic layer of said sorption material, optionally with adsorbed target compound, on the retentate site of the membrane during the recirculation of the retentate,
v) when the addition to a tangential flow filtration unit 3 is a) a composition comprising an adsorbent, adding a biological composition comprising target compound,
vi) filtrating the biological composition through the dynamic layer of adsorbent and recirculating the retentate, whereby the target compound is adsorbed by the adsorbent;
vi) adding an elution liquid to release the target compound from the sorption material, and filtrating the retentate using the tangential flow filtration unit to obtain an eluted retentate comprising the sorption material and a target-containing permeate comprising the target compound;
vi) optionally, collecting the target-containing permeate and/or the permeate of steps ii)-iv) as separated product composition(s).

6. The process according to claim 5, wherein the fluid flows of the retentate and the permeate in step iii) are adjusted such that the permeate flow is at least 5%, such as at least 10%, such as at least 15%, such as at least 20%, such as at least 25%, such as at least 30%, such as at least 35%, such as at least 40%, such as at least 45%, such as at least 50% of the retentate flow entering the tangential flow filtration unit; or in a range of 5-90%, such as 10-80%, such as 20-70%, such as 25-65%, such as 30-60% of the retentate flow entering the tangential flow filtration unit.

7. The process according to claim 5, wherein the tangential fluid flow is a continuous tangential fluid flow.

8. The process according to claim 5, comprising introducing a partial vacuum on the permeate side of the membrane, optionally, wherein
said partial vacuum results in a pressure on the permeate side in the range of 0.10 to 0.98 bar, such as in the range of 0.20 to 0.95 bar, such as in the range of 0.30 to 0.95 bar, such as in the range of 0.50 to 0.95 bar, such as in the range of 0.60 to 0.95 bar, such as in the range of 0.70 to 0.95 bar, such as in the range of 0.75 to 0.90 bar; and/or
a drainage of liquid from the permeate side of the membrane such that at least 50%, such as at least 60%, such as at least 70%, such as at least 75%, such as at least 80%, such as at least 85%, such as at least 90%, such as at least 95% of the space on the permeate side of the tangential flow filtration unit is filled with air.

9. The process according to claim 7, wherein the tangential fluid flow is a continuous tangential fluid flow.

10. The process according to claim 7, comprising introducing a partial vacuum on the permeate side of the membrane, optionally, wherein
said partial vacuum results in a pressure on the permeate side in the range of 0.10 to 0.98 bar, such as in the range of 0.20 to 0.95 bar, such as in the range of 0.30 to 0.95 bar, such as in the range of 0.50 to 0.95 bar, such as in the range of 0.60 to 0.95 bar, such as in the range of 0.70 to 0.95 bar, such as in the range of 0.75 to 0.90 bar; and/or
a drainage of liquid from the permeate side of the membrane such that at least 50%, such as at least 60%, such as at least 70%, such as at least 75%, such as at least 80%, such as at least 85%, such as at least 90%, such as at least 95% of the space on the permeate side of the tangential flow filtration unit is filled with air.
